(19) United States
(12) Patent Application Publication
(10) Pub. No.: US 2026/0102207 A1
(43) Pub. Date: Apr. 16, 2026

(54) CENTRALIZED AND MODULAR CONFIGUATION FOR UROLOGY OPERATING ROOM

(71) Applicant: Boston Scientific Scimed, Inc., Maple Grove, MN (US)

(72) Inventors: Jeffrey A. Meganck, N Grafton, MA (US); Niraj Prasad Rauniyar, Plymouth, MN (US); Andrea Marie Cahill, Cambridge, MA (US); Elizabeth A. Stokley, Raleigh, NC (US); Anant Vaidyanathan Subramaniam, Boston, MA (US); Troy Velazquez, Salem, NH (US); Kimberly DeGraaf, Holden, MA (US); Adam Perry Nodiff, Southborough, MA (US); Luke J. Hart, Westford, MA (US); Sarika Saran, Milford, MA (US); Andrew David Merrill, West Boylston, MA (US); James Mullally, Holden, MA (US)

(73) Assignee: Boston Scientific Scimed, Inc., Maple Grove, MN (US)

(21) Appl. No.: 19/357,830

(22) Filed: Oct. 14, 2025

Related U.S. Application Data
(60) Provisional application No. 63/707,003, filed on Oct. 14, 2024.

(51) Int. Cl. A61B 18/26; A61B 1/307; A61G 13/00

(52) U.S. Cl. CPC A61B 18/26; A61B 1/307; A61G 13/00; A61B 2560/0271; A61B 2560/0493

(57) ABSTRACT

The disclosure provides a centralized urology operating room control system. The centralized urology operating room control system comprising: therapy consoles selected from a group consisting of a prostate treatment console, a stone treatment console, a tabletop laser therapy console, and an endoscope control console; and a centralized urology control assembly comprising: a housing configured to house at least: a communications console comprising a speaker; a centralized controller, wherein the centralized controller includes a memory storing non-transitory instructions and a hardware processor that is configured to execute the instructions to cause display of a single user interface, via a display, at least for monitoring operation of the therapy consoles; and a power input console configured to provide single-source power to at least the communications console and the centralized controller.

### CROSS REFERENCE TO RELATED APPLICATIONS

This application claims the benefit of priority under 35 U.S.C. § 119 to U.S. Provisional Ser. No. 63/707,003 , filed Oct. 14, 2024, each of which is herein incorporated by reference in its entirety.

### TECHNICAL FIELD

The disclosure generally relates to urology operating rooms and particularly to a centralized and modular configuration within a urology operating room.

### BACKGROUND

Modern urological operating rooms employ sophisticated medical equipment systems to perform complex therapeutic procedures on patients. These operating environments typically include multiple independent technological platforms, each designed for specific clinical applications. Advanced urological procedures commonly utilize endoscopic visualization systems for minimally invasive access to anatomical structures, laser energy delivery systems for tissue ablation and fragmentation, fluidics management systems for irrigation and pressure control, and various therapeutic devices for tissue manipulation and removal.

Contemporary urological equipment encompasses a range of capital systems including prostate treatment consoles such as morcellators, stone treatment consoles like lithotripters, tabletop and floor-standing laser therapy systems, endoscope control consoles, and fluidics control equipment. These systems typically operate as standalone units, each with dedicated power sources, user interfaces, communication protocols, and display systems. The technical architecture of modern urological operating rooms reflects the independent development of these various technologies, with each system designed to fulfill specific therapeutic requirements while maintaining compatibility with standard operating room infrastructure including electrical power distribution, data networking, and environmental controls.

The complexity of urological procedures has increased in correspondence with the advancement of available technologies, requiring coordination between multiple equipment systems to achieve desired clinical outcomes. Modern urological operating room suites may include multiple rooms such as operating theaters, observation rooms, and remote control facilities, each requiring appropriate equipment provisioning and connectivity to support collaborative medical procedures.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The disclosure provides centralized and modular configurations for urology operating rooms that consolidate multiple pieces of surgical equipment to address space limitations and equipment proliferation challenges. The disclosure encompasses various architectural approaches including centralized control towers, integrated therapy systems, and modular configurations that combine equipment such as prostate treatment consoles, stone treatment consoles, laser therapy systems, endoscope control consoles, fluidics equipment, and communication systems into unified platforms. These configurations can be implemented as rack/tower systems, integrated into surgical tables or patient beds, built into operating room infrastructure including underfloor installations, or incorporated into mobile surgical bed designs. The centralized systems feature single user interfaces, centralized control tablets, communication hubs with speakers and microphones, power management systems, and cable management solutions to reduce equipment footprints. The centralized systems provide improved space utilization, noise management, electromagnetic interference reduction, and enhanced visibility for medical personnel. The disclosure describes twelve distinct embodiments ranging from basic centralized towers with separate floor-standing equipment to fully integrated surgical beds with built-in therapy consoles and comprehensive underfloor control systems, providing scalable solutions for modernizing urology operating room configurations across various applications and environmental contexts.

In some embodiments, the disclosure can be implemented as a centralized urology operating room control system comprising: a plurality of therapy consoles selected from a group consisting of a prostate treatment console, a stone treatment console, a tabletop laser therapy console, and an endoscope control console; and a centralized urology control assembly configured to house at least: a communications console comprising a speaker; a centralized controller, wherein the centralized controller includes a memory storing non-transitory instructions and a hardware processor that is configured to execute the instructions to cause display of a single user interface, via a display, at least for monitoring operation of the plurality of therapy consoles; and a power input console configured to provide single-source power to at least the communications console and the centralized controller.

With further embodiments, the centralized urology operating room control system can comprise wherein the stone treatment console comprises a single lithotripsy system incorporating mechanical, laser, and suction capabilities.

With further embodiments, the centralized urology operating room control system can comprise wherein the plurality of therapy consoles are housed in the centralized urology control assembly.

With further embodiments, the centralized urology operating room control system can comprise wherein the centralized urology control assembly includes a noise cancelling console configured to cancel operational noise associated with operation of one or more of the plurality of therapy consoles.

With further embodiments, the centralized urology operating room control system can comprise wherein the centralized urology control assembly houses fluidics control equipment.

With further embodiments, the centralized urology operating room control system can comprise wherein the plurality of therapy consoles are housed in a urology treatment assembly that is separate from the centralized urology control assembly, wherein the urology treatment assembly includes an endoscope control console and a therapy communication console.

With further embodiments, the centralized urology operating room control system can comprise wherein the urology treatment assembly is comprised of a plurality of therapy-specific urology treatment assemblies which are each configured to house a respective therapy console of the plurality of therapy consoles.

With further embodiments, the centralized urology operating room control system can comprise wherein each of the therapy-specific urology specific assemblies are configured to as communicatively couple, physically couple, or both physically and communicatively couple to a floor-standing laser.

With further embodiments, the centralized urology operating room control system can comprise wherein each of the therapy-specific urology specific assemblies are configured to communicatively couple, physically couple, or both physically and communicatively couple to floor-standing fluidics equipment, and wherein each of the therapy-specific urology specific assemblies includes a table-top laser console.

With further embodiments, the centralized urology operating room control system can comprise wherein the urology treatment assembly is a centralized urology treatment assembly configured to house each of the plurality of therapy consoles.

With further embodiments, the centralized urology operating room control system can comprise wherein the centralized urology treatment assembly, the centralized urology control assembly, or both, are configured to be housed within a recess in an operating room structure, wherein the operating room structure is a wall of the operating room, a ceiling of the operating room, a floor of the operating room, a patient bed, or a patient table.

With further embodiments, the centralized urology operating room control system can comprise the single-user interface is configured to concurrently display real-time information associated with two or more of the plurality of therapy consoles via the display; and the display is a built-in display, a remote display, or both the built in display and the remote display.

With further embodiments, the centralized urology operating room control system can comprise a surgical navigation console configured to provide: real-time tracking of at least one of medical devices, body parts, or device motion during urological procedures; and cause display, via the display, of a representation of the real-time position of the at least one of medical devices, body parts, or device motion during urological procedures.

With further embodiments, the centralized urology operating room control system can comprise wherein the communications console is configured to provide a wired communication capability, a wireless communication capability, or both, with a mobile computing device, wherein the mobile computing device is configured to remotely monitor, control operation of, or monitor and control operation of one or more of the plurality of therapy consoles.

With further embodiments, the centralized urology operating room control system can comprise wherein the communications console includes a microphone, and wherein the centralized processor is configured to control operation of one or more of the plurality of therapy consoles via voice commands received via the microphone.

In some embodiments, the disclosure can be implemented as a centralized urology operating room control system comprising: a plurality of therapy consoles selected from a group consisting of a prostate treatment console, a stone treatment console, a tabletop laser therapy console, and an endoscope control console; an access floor positioned above and spaced a distance from a sub-floor to create an inner floor space between the access floor and the sub-floor; a centralized urology control assembly disposed in the inner floor space, the centralized urology control assembly comprising: a housing defining a plurality of receptacles which collectively house at least: a power input console providing electrical power to said plurality of therapy consoles; a communications console comprising a speaker; and a centralized processor, wherein the centralized processor is configured to provide a single user interface, via a built-in display, for controlling operation of at least one of the plurality of therapy consoles; and a mobile computing device that is communicatively coupled to and configured to remotely control the plurality of therapy consoles, the centralized urology control assembly, or both the plurality of therapy consoles and the centralized urology control assembly.

With further embodiments, the centralized urology operating room control system can comprise wherein the plurality of therapy consoles are housed in a centralized urology treatment assembly that is separate from the centralized urology control assembly, wherein the centralized urology treatment assembly includes an endoscope control console and a communication console.

With further embodiments, the centralized urology operating room control system can comprise wherein the centralized urology treatment assembly is disposed in the inner floor space.

With further embodiments, the centralized urology operating room control system can comprise wherein the centralized urology treatment assembly is a free-standing movable tower on and movable about the access floor.

With further embodiments, the centralized urology operating room control system can comprise wherein centralized urology treatment assembly is disposed within a recess in a surgical table.

With further embodiments, the centralized urology operating room control system can comprise wherein the plurality of therapy consoles are housed in a therapy-specific urology treatment assemblies that is separate from the centralized urology control assembly, wherein the therapy-specific urology treatment assemblies includes an endoscope control console and a communication console, and wherein each of the therapy-specific urology specific treatment assemblies are configured to communicatively couple to a laser, wherein the laser is a floor-standing laser or a tabletop laser.

With further embodiments, the centralized urology operating room control system can comprise: a plurality of floor plugs in the access floor that are associated with the centralized urology control assembly, wherein the plurality of floor plugs are selected from the group consisting of display and data floor plugs, electrical device floor plugs, and optomechanical device floor plugs; a plurality of protective covers configured to cover the plurality of floor plugs; and a floor drain system located in the access floor that is configured to route fluid away from the centralized urology control assembly, the plurality of floor plugs, or both.

In some embodiments, the disclosure can be implemented as a centralized urology operating room control system comprising: a plurality of therapy consoles selected from a group consisting of a prostate treatment console, a stone treatment console, a tabletop laser therapy console, and an endoscope control console; a patient support apparatus configured to physically support a patient located on the patient support apparatus, wherein the patient support apparatus includes a recess configured to receive at least the plurality of therapy consoles; and a centralized urology control assembly, the centralized urology control assembly comprising: a housing defining a plurality of receptacles which collectively house at least: a power input console providing electrical power to said plurality of therapy consoles; a communications console comprising a speaker; and a centralized processor, wherein the centralized processor is configured to provide a single user interface, via a built-in display, for controlling operation of at least one of the plurality of therapy consoles.

With further embodiments, the centralized urology operating room control system can comprise wherein the patient support apparatus is a patient table or a patient bed.

With further embodiments, the centralized urology operating room control system can comprise wherein the centralized urology control assembly is configured to be disposed in the recess in the patient support apparatus or is configured to be coupled to an exterior surface of the patient support apparatus.

### BRIEF DESCRIPTION OF THE DRAWINGS

To easily identify the discussion of any element or act, the most significant digit or digits in a reference number refer to the figure number in which that element is first introduced.

FIG. 1A illustrates a first operating room (OR) environment in accordance with at least one embodiment.

FIG. 1B illustrates a centralized operating theater controller of the first OR environment of FIG. 1A in greater detail.

FIG. 2A, FIG. 2B, FIG. 2C, FIG. 2D, FIG. 2E, and FIG. 2F illustrate a second OR environment, provisioned for a urological procedure, in accordance with at least one embodiment.

FIG. 3A illustrates a third OR environment in accordance with at least one embodiment.

FIG. 3B illustrates a centralized operating theater controller of the third OR environment of FIG. 3A in greater detail.

FIG. 4 illustrates a first centralized urology operating room control system in accordance with at least one embodiment.

FIG. 5 illustrates a second centralized urology operating room control system in accordance with at least one embodiment.

FIG. 6 illustrates a third centralized urology operating room control system in accordance with at least one embodiment.

FIG. 7 illustrates a fourth centralized urology operating room control system in accordance with at least one embodiment.

FIG. 8 illustrates a fifth centralized urology operating room control system in accordance with at least one embodiment.

FIG. 9 illustrates a sixth centralized urology operating room control system in accordance with at least one embodiment.

FIG. 10 illustrates a seventh centralized urology operating room control system in accordance with at least one embodiment.

FIG. 11 illustrates an eighth centralized urology operating room control system in accordance with at least one embodiment.

FIG. 12 illustrates a ninth centralized urology operating room control system in accordance with at least one embodiment.

FIG. 13 illustrates a tenth centralized urology operating room control system in accordance with at least one embodiment.

FIG. 14 illustrates an eleventh centralized urology operating room control system in accordance with at least one embodiment.

FIG. 15 illustrates a twelfth centralized urology operating room control system in accordance with at least one embodiment.

FIG. 16 illustrates a computer-readable storage medium in accordance with at least one embodiment.

FIG. 17A illustrates a first logic flow in accordance with at least one embodiment.

FIG. 17B illustrates a second logic flow in accordance with at least one embodiment.

### DETAILED DESCRIPTION

The present disclosure is described with reference to medical devices, methods, and systems. Often, the disclosure is described with reference to surgical urological equipment and procedures. For example, in some procedures, a medical device (e.g., an endoscope, a laser fiber, a snare, a basket, etc.) may be advanced through a path or passage in a body (e.g., a ureter) to aid in removal of target tissue (e.g., a stone, or the like) from a cavity in the body (e.g., a calyx of a kidney). In another example, a medical device (e.g., an endoscope, a laser fiber, a morcellator, etc.) may be advanced through a path or passage in a body (e.g., a ureter) to aid in treatment and/or removal of target tissue (e.g., cancerous prostate tissue, or the like).

It is to be appreciated that references to a particular type of procedure, medical device, target tissue, or body passage or cavity are provided for convenience and clarity of describing the invention and are not intended to limit the claims beyond what is specified in each claim.

The terms “proximal” and “distal” may be utilized along with terms such as “parallel,” “transverse,” and “longitudinal” to describe the relative relationship and position of elements described herein. Proximal refers to a position closer to the exterior of the body (or closer to a user), whereas distal refers to a position closer to the interior of the body (or further away from the user). Further, the term “elongated” as used herein refers to an object that is substantially longer in one direction (e.g., referred to as the longitudinal direction) in relation to a perpendicular direction. For example, an object having a longer width than length could be referred to herein as elongated.

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its structure and method, together with further objects and advantages will be better understood from the following description when read in conjunction with the accompanying figures.

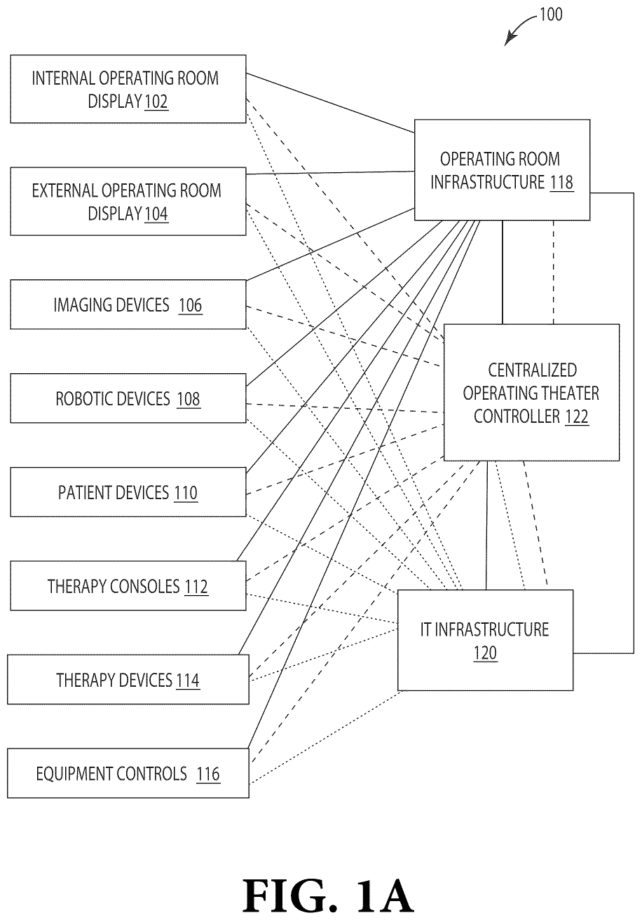

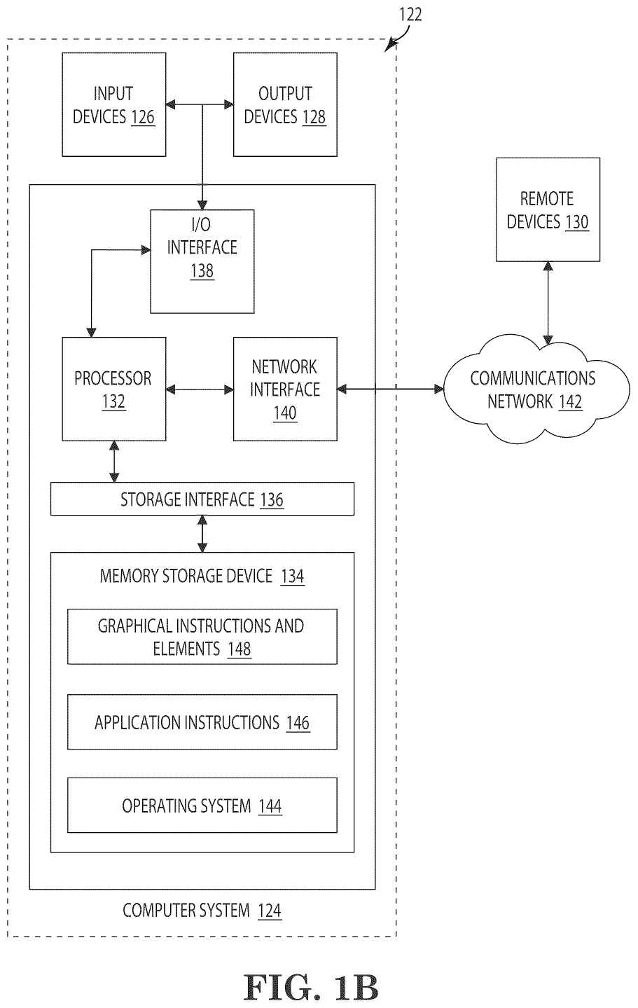

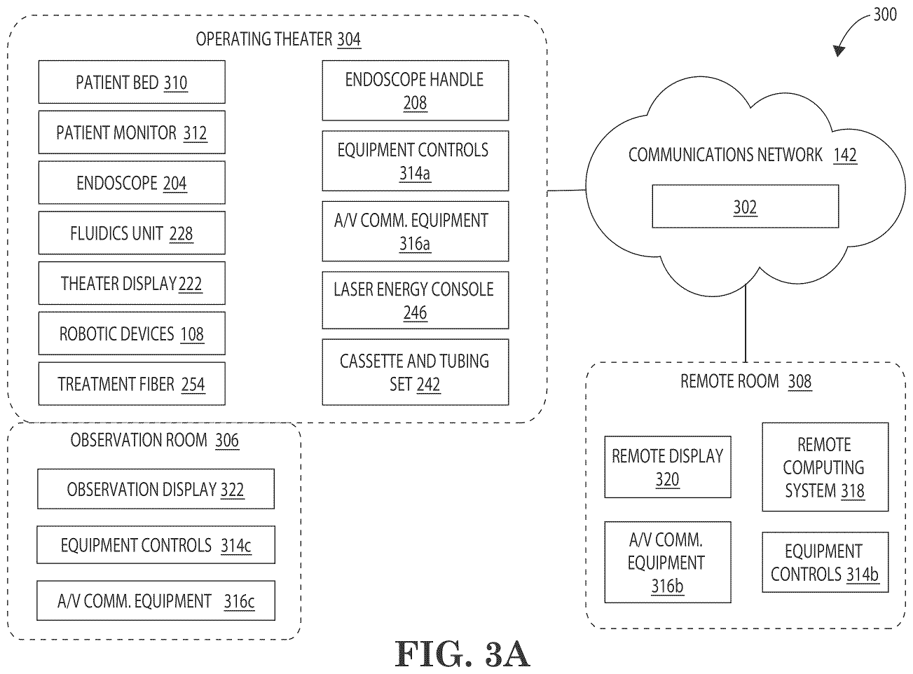

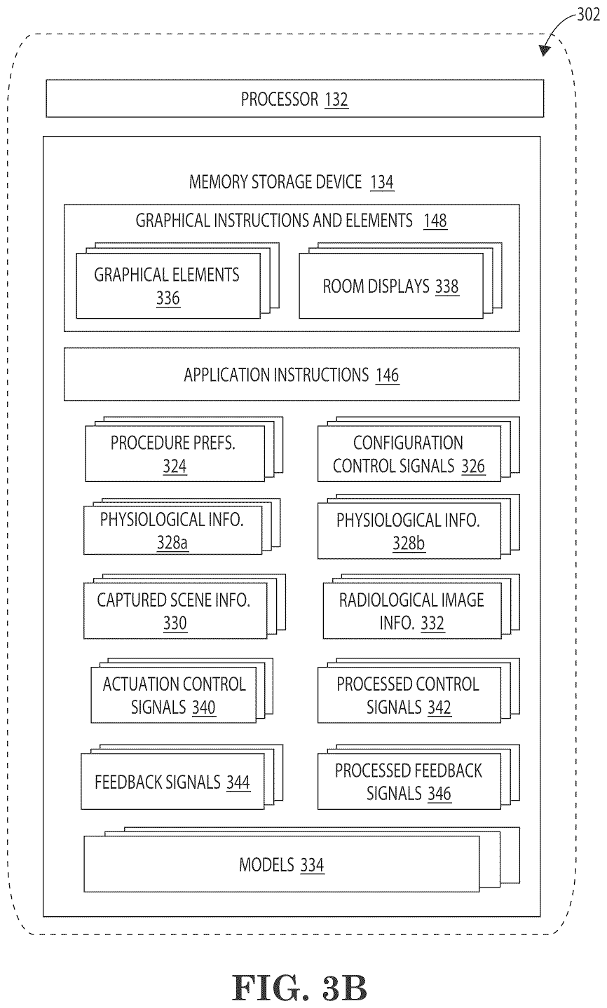

Numerous aspects of the present disclosure are now described with reference to an illustrative operating room (OR) environment 100 as depicted in FIG. 1A. The OR environment 100 will often be focused on an operating theater (or surgical suite), which is a designated room or space where the patient and procedure take place. However, the OR environment 100, and particularly, the equipment of OR environment 100 can be disposed and/or used in multiple locations including the operating theater (e.g., see FIG. 3A). Locations outside or other than the operating theater can include a viewing room, a control room, a computing infrastructure room, a proctor viewing room, a remote physician operating room, or the like). As will be appreciated, not all locations in which equipment of OR environment 100 will be deployed are sterile. Further, even in the operating theater itself there is often a delineation between a sterile and non-sterile field.

OR environment 100 can include internal and external displays. For example, OR environment 100 can include an internal operating room display 102 (or displays) arranged to display information within the operating theater. Similarly, OR environment 100 can include external operating room display 104 (or displays) arranged to display information outside the operating theater, for example, to observers, to remote physicians, to proctors, to nurses or admins outside the sterile field or in another room. Further, OR environment 100 can include imaging devices 106, robotic devices 108, patient devices 110, therapy consoles 112, therapy devices 114, equipment controls 116, operating room infrastructure 118, information technology (IT) infrastructure 120, and centralized operating theater controller 122.

With some embodiments, internal operating room display 102 can include an overhead operating theater display. As a specific example, internal operating room display 102 can include a display physically located inside of the operating theater (e.g., a wall mounted monitor, a ceiling mounted monitor, a monitor mounted on an articulating arm, or the like) that is visible to multiple observers. In some embodiments, internal operating room display 102 can include a wearable display, such as, a heads-up display, a virtual reality display, an augmented reality display, a tablet or small form factor display and associated harness to wear the display. With some embodiments, multiple wearable displays can be provided. For example, the physician can wear a head mounted display while an assistant wears a tablet computer. In some embodiments, internal operating room display 102 can include an on-patient display, for example, a display or monitor physically attached to the patient, a display projected onto the patient, or the like.

In some embodiments, external operating room display 104 can include a monitor or monitors located outside the operating theater and configured to display information from inside the operating theater to a person outside the operating theater. For example, external operating room display 104 can include a monitor or monitors configured to display a view (or views) of the surgical suite. In some examples, the external operating room display 104 can be located proximate to (e.g., in the next room, or the like) the surgical suite while in other examples, the external operating room display 104 is in another area of the premises, off premises (e.g., in another geographical location, or the like). In some embodiments the external operating room display 104 can be configured for direct real-time viewing, recorded viewing, or both. With some embodiments, external operating room display 104 can include a monitor or monitors configured to display information from the surgical suite or components of the OR environment 100. For example, external operating room display 104 could be a monitor configured to mirror the displayed contents on a monitor associated with another piece of equipment in the surgical suite (e.g., vital monitoring equipment, therapy console, or the like). It is noted that equipment provisioned in the OR environment 100 can have its own display. Examples of these displays are described herein and can be classified as internal operating room display 102 or external operating room display 104 depending upon the location of the equipment.

Imaging devices 106 can include any of a variety of imaging devices configured to capture images of the patient, either pre-procedure, intra-procedure, or post-procedure. The imaging devices 106 can utilize any of several imaging modalities (e.g., radiography, ultrasound, tomographic, direct visualization, or the like). With some embodiments, imaging devices 106 can include a planar X-ray device, a fluoroscopy device, or the like. In some embodiments, imaging devices 106 can include an ultrasound imaging device. In some embodiments, imaging devices 106 can include a magnetic resonance imaging (MRI) device, a computed tomography (CT) scanning device, a positron emission tomography (PET)-MRI device, single-photon emission (SPE)-CT scanning device, or the like.

Robotic devices 108 can include any of a variety of robotic equipment configured to automatically or under control of a user, observe and/or assist in the procedure. For example, the robotic devices 108 can be equipment configured to provide a surgical navigation system (e.g., device, motion, body part tracking, or the like) and computing resources (e.g., processing circuitry, memory, etc.) configured to provide real-time tracking (e.g., needle tracking, therapy device tracking, or the like) or something in the operating theater (e.g., a body part, a medical device, or the like). In some examples, robotic devices 108 can include motion control, articulation, grasping to facilitate automatic movement, analysis, or control of equipment in the OR environment 100. Additionally, robotic devices can be configured to manipulate ones of the devices (e.g., therapy devices 114, or the like) automatically and/or under remote or non-contact control from a physician.

Patient devices 110 can include any equipment in direct contact with the patient, for example, anesthesia equipment, vital monitoring equipment, the surgical bed, and surgical bed accessories. With some embodiments, patient devices 110 can include equipment to provide general or local anesthesia to the patient, such as, a continuous-flow anesthetic machine, or the like. As another example, patient devices 110 can include continuous bedside monitors (e.g., for temperature, pulse, etc.), hemodynamic monitors, respiratory monitors, neurological monitors, cardiac monitors, or the like.

Therapy consoles 112 and therapy devices 114 can comprise any equipment (e.g., capital equipment, single use devices, reusable devices, or the like) arranged to perform or provide the treatment associated with the procedure. In general, therapy consoles 112 can include any capital equipment and/or infrastructure used for delivery of the desired treatment or therapy. For example, 112 can include visualization equipment, such as, endoscope viewing and/or control consoles. As another example, therapy consoles 112 can include fluidic consoles to provide fluid inflow and/or outflow, suction, or the like. In another example, therapy consoles 112 can include lithotripsy equipment, such as, laser consoles configured to generate laser energy to ablate, fragment, dust, or otherwise treat calculi. With another example, therapy consoles 112 can include soft tissue therapy equipment, such as, morcellation consoles, ablation consoles, resection consoles, biopsy consoles, cauterization consoles (e.g., laser, electrocautery, radio frequency (RF) cautery, or the like).

Therapy devices 114 can include any device used with the therapy consoles 112 to affect the treatment or procedure. For example, therapy devices 114 can include endoscopes, such as, a ureteroscope, a cystoscope, a nephroscope, or a resectoscope. The endoscopes can be electronic or optical and can be configured to visualize the anatomy in minimally invasive procedures. With some examples, therapy devices 114 can include retrieval devices (e.g., baskets, snares, loops, hooks, pinchers, or the like). In some examples, therapy devices 114 can include optical fibers to convey laser energy to a treatment site, morcellation devices, energy delivery devices (e.g., thermal, electric, RF, or the like). In some examples, therapy devices 114 can include post-procedure or healing devices, such as, stents and stent delivery devices, or the like. Any of the therapy devices 114 can be single use devices, reusable devices, or therapy devices 114 can include a combination of single use and reusable devices.

Equipment controls 116 includes all equipment and/or interfaces used to control equipment in OR environment 100 and/or facilitate exchange of data between equipment in OR environment 100. Examples of such controls are provided throughout this disclosure.

Operating room infrastructure 118 can include any equipment built into the physical infrastructure of the surgical suite. For example, operating room infrastructure 118 can include operating theater lighting (e.g., wall mounted, ceiling mounted, mounted to an articulating arm, or the like) configured to provide illumination of the room and/or surgical field. In some examples, operating room infrastructure 118 can include image and/or audio capture devices (e.g., video cameras, or the like). In some examples, operating room infrastructure 118 can include centralized air, water, and/or gas supply lines (e.g., suction, filtered water, oxygen, etc.) In some examples, operating room infrastructure 118 can include central waste collection systems (e.g., floor drain, or the like). In some examples, operating room infrastructure 118 can include warming systems (e.g., warming oven, or the like) configured to warm consumables used during a procedure. With some examples, operating room infrastructure 118 can include electrical power supplies and can include hardwired or mobile power supplies.

IT infrastructure 120 can include any structure and equipment used for the transmission, storage, and/or processing of data used in the procedure. For example, IT infrastructure 120 can include servers, data storage arrays, data centers, wire and/or wireless communication cabling and equipment, medical record data storage devices, or the like.

As depicted, the components of OR environment 100 are coupled to centralized operating room infrastructure 118 and IT infrastructure 120. For example, internal operating room display 102, external operating room display 104, imaging devices 106, robotic devices 108, patient devices 110, therapy consoles 112, and/or therapy devices 114 can be configured to receive and/or transmit data (e.g., information elements, control signals, etc.) via IT infrastructure 120. Such exchange of data can be unidirectional or bidirectional. Examples of this are provided throughout the disclosure.

Further, components of OR environment 100 that contain electrical and/or electromechanical elements can be coupled to a source of power via operating room infrastructure 118, Similarly, components of the OR environment 100 may be coupled to fluid and/or gas supplies via operating room infrastructure 118. For example, internal operating room display 102, external operating room display 104, imaging devices 106, robotic devices 108, patient devices 110, therapy consoles 112, and/or therapy devices 114 can be configured to receive electrical power, gas supply, water inflow and/or outflow supply, vacuum supply, or the like in any combination via operating room infrastructure 118.

Lastly, centralized operating theater controller 122 can be coupled to any equipment or components of the OR environment 100 via IT infrastructure 120. FIG. 1B illustrates an example centralized operating theater controller 122. In general, the centralized operating theater controller 122 can be configured to send and/or receive data to and/or from equipment (e.g., therapy consoles internal operating room display 102, external operating room display 104, imaging devices 106, robotic devices 108, patient devices 110, therapy consoles 112, therapy devices 114, equipment controls 116, and/or operating room infrastructure 118).

Further, centralized operating theater controller 122 can be configured to process data, infer information from data, and execute instructions to implement methods described herein.

It is noted that centralized operating theater controller 122 is depicted in FIG. 1A and FIG. 1B as a single component. However, in practice centralized operating theater controller 122 can be multiple components or a single component. Further, centralized operating theater controller 122 can be embodied (e.g., housed) in one of the other pieces of equipment depicted in FIG. 1A. For example, a one of the therapy consoles 112 can include hardware as depicted in FIG. 1B and can be configured as outlined herein to operate as centralized operating theater controller 122.

As depicted in FIG. 1B, centralized operating theater controller 122 can include computer system 124, input devices 126, output devices 128, and/or remote devices 130.

The computer system 124 may include processor 132 and a memory storage device 134 coupled to the processor 132 via a storage interface 136. In general, processor 132 can be processing circuitry configured to execute instructions stored on memory storage device 134. For example, processor 132 can be a central processing unit (CPU), a graphics processing unit, a machine learning (ML) processing unit, or a combination of these. The processor 132 may include specialized processing units such as integrated system (bus) controllers, memory management control units, floating point units, graphics processing units, neural processing units, digital signal processing units, etc. Processor 132 can be an off the shelf CPU or can be custom designed processing circuitry (e.g., an application specific integrated circuit (ASIC), or the like).

Memory storage device 134 may include computer-readable storage media or devices configured to store data. Such data can take a variety of forms or data structures. One form of such data is machine code (also referred to as “instructions”) that is executable by processor 132. In some examples, memory storage device 134 can be physical memory on which information or data readable by a processor (e.g., processor 132) may be stored. Thus, a computer-readable storage medium may store instructions for execution by one or more processors (e.g., processor 132, or the like) including instructions for causing the processor(s) to perform steps or stages consistent with the embodiments described herein.

The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media. Although depicted in FIG. 1B, memory storage device 134 need not be collocated with processor 132 and can for example, be accessible via a network.

In some embodiments, the storage interface 136 may be configured to connect to memory storage device 134 via memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Processor 132 can be disposed in communication with input devices 126 and output devices 128 via I/O interface 138. The I/O interface 138 may employ communication protocols and/or methods such as, without limitation, audio, analog, digital, stereo, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), Ethernet, Bluetooth, cellular, etc. Using the I/O interface 138, computer system 124 may communicate with input devices 126 and output devices 128. In general, input devices 126 can be any control or input device used to provide input to equipment in the OR environment 100.

In some embodiments, input devices 126 can include devices configured to receive input from a user of the OR environment 100 (e.g., a physician, a nurse, an assistant, a technician, or the like). For example, input devices 126 can include wearable controls such as watches, armbands, headphones, footwear, or the like. In another example, input devices 126 can include touch-based controls such as foot pedals, switches, triggers, touch screens, buttons, or the like. In some examples, input devices 126 can include non-touch-based controls such as voice activation controls, gesture based controls, eye movement based controls, or the like. These various input devices implemented as input devices 126 can be equipment controls 116 described above or can be inputs to other computing equipment (e.g., imaging devices 106, robotic devices 108, patient devices 110, therapy consoles 112, therapy devices 114, etc.)

With some embodiments, output devices 128 can include internal operating room display 102 and/or external operating room display 104. With some embodiments, output devices 128 can include non-display outputs such as audio output, flashing light output, haptic output, or the like. Further, in some examples, ones of input devices 126 and/or output devices 128 can be combined. For example, a touch screen display can be configured as both input devices 126 and output devices 128.

It is to be appreciated that although input devices 126 and output devices 128 are depicted as included (or packaged) with computer system centralized operating theater controller 122, they may not be explicitly part of centralized operating theater controller 122 but could be I/O devices of another computer system described herein with which centralized operating theater controller 122 is configured to communicate.

As noted, memory storage device 134 may store instructions executable by processor 132. This can include various types of instructions. For example, memory storage device 134 can store an operating system 144 and/or application instructions 146. Further, memory storage device 134 can store graphical instructions and elements 148 (e.g., user interface elements, graphical information elements, etc.) In various embodiments, the operating system 144 may facilitate resource management and operation of the computer system 124 and facilitate communicative coupling with input devices 126, output devices 128, and other equipment in the OR environment 100 coupled via IT infrastructure 120. Examples of operating systems include, without limitation, UNIX®, UNIX-like system distributions (e.g., BERKELEY SOFTWARE DISTRIBUTION® (BSD), FREEBSD®, NETBSD®, OPENBSD, etc.), LINUX® DISTRIBUTIONS (e.g., RED HAT®, UBUNTU®, KUBUNTU®, etc.), IBM® OS/2®, MICROSOFT® WINDOWS®, APPLE® IOS®, GOOGLE™ ANDROID™, or the like.

The application instructions 146 may include instructions that when executed by the processor 132 cause centralized operating theater controller 122 to perform one or more techniques, steps, procedures, and/or methods described herein, such as to send and/or receive data to and/or from equipment of OR environment 100, process the data, infer information from ML models, execute algorithms based on the data, and send and/or receive control signals to and/or from the equipment in the OR environment 100.

The graphical instructions and elements 148 may include instructions that when executed by the processor 132 cause the processor 132 to facilitate rendering and display of information on displays in patient devices 110. The graphical instructions and elements 148 can also include the rendered graphical elements (or frames) to be displayed. For example, graphical instructions and elements 148 can be configured to provide cursors, icons, checkboxes, menus, scrollers, windows, widgets, etcetera. Such graphical instructions and elements 148 can provide an interface (e.g., analog, or digital) to equipment of the OR environment 100 or a display of settings, parameters, status, or other information related to the equipment.

In some embodiments, the processor 132 may be in communication with remote devices 130 via network interface 140 and communications network 142. Remote devices 130 can be any of a variety of computing devices (e.g., cloud computing resources, cloud storage resources, remote servers, remote sensors, remote workstations, etc.) The network interface 140 may permit communication between computer system 124 and remote devices 130 via the communications network 142. To that end, the network interface 140 may employ connection protocols including, without limitation, direct connect, Ethernet, Transmission Control Protocol/Internet Protocol (TCP/IP), token ring, Wi-Fi, etc. The communications network 142 can be implemented as one of the different types of networks, such as intranet or Local Area Network (LAN), Closed Area Network (CAN), the Internet, and such. The communications network 142 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 142 may include a variety of network devices, including routers, bridges, servers, computing devices, storage devices, etcetera.

Many pieces of equipment in OR environment 100 can include computing components or a computing system. For example, therapy consoles 112 often include computing systems including computing components, like those described above, with respect to centralized operating theater controller 122 and particularly computer system 124. Accordingly, computing components of such devices are often discussed with reference to the components of computer system 124. However, this is done for convenience only. It is to be appreciated that these other computer systems may include some or all the components of computer system 124 and may include different components or additional components that are not shown in FIG. 1B. In general, however, such computer system will include a processor (e.g., like processor 132) and a memory storage device (e.g., like memory storage device 134) that includes instructions.

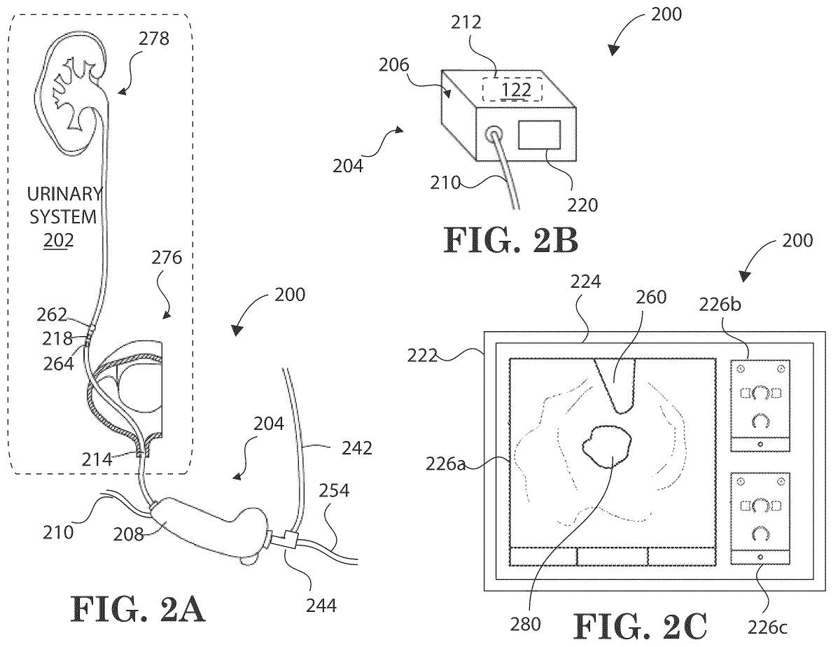

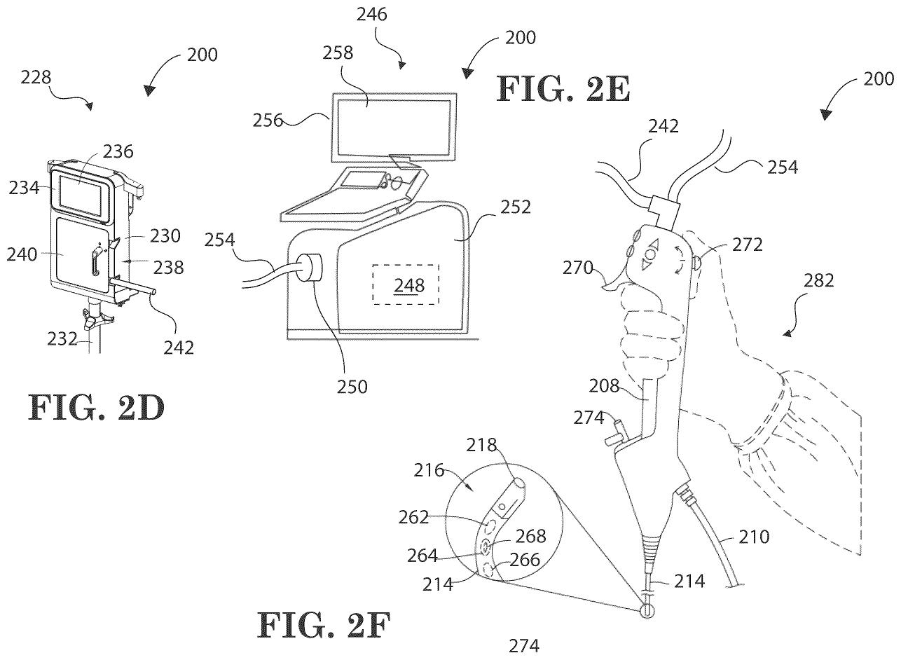

As noted above, OR environment 100 can be provisioned and implemented to perform urological procedures. For example, FIG. 2A to FIG. 2E illustrates an OR environment 200, which can be implemented using components from OR environment 100 described above to perform a urological procedure. OR environment 200 is described with respect to a lithotripsy procedure to treat urinary calculi (referred to as “stone”) in a urinary system 202. However, this is not intended to be limiting and OR environment 100 and/or OR environment 200 could be implemented to perform other urological procedures, such as, for example, percutaneous nephrolithotomy (PCNL), benign prostatic hyperplasia (BPH), transurethral resection of the prostate (TURP), etc.

OR environment 200 can be implemented with an endoscope 204, such as, a ureteroscope. Endoscope 204 can include an endoscope console 206 (e.g., one of therapy consoles 112), which can be configured to operate with an endoscope handle 208 (e.g., one of therapy devices 114). The endoscope console 206 and endoscope handle 208 can be coupled via connection cable 210. FIG. 2A, and FIG. 2F illustrate endoscope handle 208 in OR environment 200 while FIG. 2B illustrates endoscope console 206. Endoscope 204 can be coupled to a source of power via operating room infrastructure 118. Further, the connection cable 210 can be configured to provide power from endoscope console 206 to endoscope handle 208 and to provide exchange of data between endoscope console 206 and endoscope handle 208.

Endoscope console 206 can include computing system 212 (e.g., like computer system 124) which itself can include (or be coupled to) a display 220 (e.g., touch screen display, or the like). Endoscope console 206 can also include input and/or output devices (not shown), such as buttons, lights, switches, etc. Further, OR environment 200 can include computing components configured to operate as the centralized operating theater controller 122 described above with respect to FIG. 1A and FIG. 1B. With some embodiments, as shown, centralized operating theater controller 122 can be integrated into one of the therapy consoles 112. Accordingly, endoscope console 206 can include computing system 212 and computer system 124.

In some embodiments a single computing system (e.g., computer system 124, or the like) can be provided as part of endoscope console 206 and configured to operate as both computing system 212 and centralized operating theater controller 122. For example, FIG. 2B illustrates centralized operating theater controller 122 into integrated endoscope console 206 as part of computing system 212. However, this is not intended to be limiting and centralized operating theater controller 122 could be a separate computing system disposed in the housing of endoscope console 206 or could be integrated into another therapy consoles 112 provisioned in the OR environment 200 (e.g., theater display 222, fluidics unit 228, laser energy console 246, or the like). Or as depicted in OR environment 100, centralized operating theater controller 122 of OR environment 200 could be a stand-alone component provisioned in OR environment 200. In some embodiments, centralized operating theater controller 122 can be a cloud computing system (e.g., computing as a service (CaaS), or the like) accessible via communications network 142. In such an example, equipment in OR environment 200 (e.g., computing system 212 of endoscope console 206, or the like) can include network interfaces (e.g., network interface 140) to permit communication with centralized operating theater controller 122 on communications network 142.

Operation of centralized operating theater controller 122 in OR environment 200 is described in greater detail below. However, in general, centralized operating theater controller 122 is configured to provide data communication between devices provisioned in OR environment 200. For example, centralized operating theater controller 122 can be configured to provide data communication between endoscope 204, display 220, fluidics unit 228, laser energy console 246, and their associated therapy devices 114, or any combination of these components. Further, centralized operating theater controller 122 can be configured to process such data as outlined herein (e.g., send and receive control signals between devices based on (or responsive to) the communicated data, execute algorithms on the communicated data as part of controlling operation of the components, or the like).

Endoscope 204 can include an elongated shaft 214 coupled to endoscope handle 208, which can be used to access a patient's bladder 276 and/or kidney 278. In such a procedure, the endoscope 204, and particularly, a distal end 216 of the elongated shaft 214 is inserted into the bladder 276 via the urethra and can be further inserted into the kidney 278 via the ureter, where it can be used to diagnose and/or treat a variety of problems in the urinary system 202. The endoscope 204 can include a camera 218 disposed on the distal end 216 of the elongated shaft 214. The camera 218 can be used to provide a visual feed on a display screen. For example, images captured by camera 218 can be rendered and displayed on display 220 of endoscope console 206. Additionally, OR environment 200 can be provided with several other displays (e.g., internal operating room display 102 and/or external operating room display 104) that can be configured to display images and/or video captured by camera 218 of endoscope 204. For example, centralized operating theater controller 122 can be configured, or rather, can include application instructions 146 stored in memory storage device 134 that when executed by processor 132 cause centralized operating theater controller 122 to receive data comprising indications of image frames captured by camera 218, process the image frames, and send the processed image frames to theater display 222 for display. Such communication can be facilitated by communicative connection between the equipment in OR environment 200 via IT infrastructure 120.

For example, FIG. 2C illustrates a theater display 222 (or operating theater display), in which is depicted a composite display 224 having a grouping of individual graphical elements 226a to 226c. For example, theater display 222 shows composite display 224 having graphical element 226a, 226b, and 226c where graphical element 226a depicts a view of an image captured by camera 218. It is to be appreciated that this view could be a live view or a recorded view and various examples of each are provided herein.

OR environment 200 can further include a fluidics unit 228 (e.g., as one of therapy consoles 112). Fluidics unit 228 can be coupled to endoscope 204 and called on to provide fluid flow to the distal end 216 of the elongated shaft 214. For example, fluidics unit 228 could be utilized to clear the visual field of the camera 218. Fluidics unit 228 can include a console 230. In some examples, console 230 can be mounted on pole 232 attached to a mobile base (not shown). In other examples, console 230 can be free standing, table mounted, or the like. Console 230 can include computing system 234 (e.g., like computer system 124) which itself can include a display 236 (e.g., touch screen display, or the like). Fluidics unit 228 can also include input and/or output devices (not shown), such as, buttons, lights, switches, etc.

Console 230 can include an interface (not shown) with connection sockets and/or busses to which computing system 234 can be communicatively coupled to centralized operating theater controller 122 via IT infrastructure 120. Such interface can also couple fluidics unit 228 to a source of power via operating room infrastructure 118. For example, a connection cable (not shown) could couple computing system 234 to centralized operating theater controller 122 in endoscope console 206 (e.g., via IT infrastructure 120, or the like) and couple fluidics unit 228 to power provided by operating room infrastructure 118.

Fluidics unit 228 can include a pump 238 (disposed in console 230). The pump can be configured to provide fluid flow when requested by the user (e.g., via the endoscope handle 208, or the like). Fluidics unit 228 can be configured to operate with a cassette and tubing set 242 (e.g., one of therapy devices 114, or the like). The cassette and tubing set 242 can be disposed in console 230 via door 240. Further, the cassette and tubing set 242 can be coupled to a source of fluid (not shown) and to the endoscope handle 208 (e.g., via fluid port 244 as shown in FIG. 2A, or the like). In some examples, the fluid source can be saline bags, or the like.

The computing system 234 can control pump 238 (e.g., responsive to input from endoscope 204, endoscope handle 208, responsive to sensor(s) output, responsive to control signals from centralized operating theater controller 122, or the like) to cause fluid to flow to the distal end 216 of the elongated shaft 214 via a working channel or dedicated fluid channel (not shown) in the elongated shaft 214. Additionally, in some embodiments, fluidics unit 228 can include a heater and/or a chiller to heat and/or cool the fluid supplied to the treatment site via the elongated shaft 214. Fluid flow to the treatment site (e.g., body cavity, or the like) in the urinary system 202 where the stone 280 is located affects the pressure inside the body cavity. This pressure is referred to herein as intraluminal pressure (ILP).

During an example lithotripsy procedure, blood and/or debris may be present in the body cavity, which may negatively affect image quality captured by the endoscope 204. Fluid flow (e.g., irrigation fluid flow) from fluidics unit 228 may be used to flush the body cavity to improve the image quality. Further, as laser energy (described below) can be used to fragment, ablate, dust, or otherwise treat the stone, heat may be generated at the treatment site. Fluid flow can be used to control the temperature of the treatment site to avoid damage or injury to adjacent tissue.

OR environment 200 can further include a laser energy console 246 (e.g., as one of therapy consoles 112) provisioned in OR environment 200. Continuing with the example discussed above where OR environment 200 is provisioned for a lithotripsy procedure, laser energy console 246 could be a medical laser console, such as, a Holmium (Ho) laser or a Thulium (Tm) fiber laser console. As another example, laser energy console 246 could be a tissue ablation console (e.g., electronic ablation, RF ablation, etc.). With yet another example, laser energy console 246 could be a laser morcellator. With yet another embodiment, the laser energy console 246 can be a green light laser configured to generate green light and including a fiber optic delivery system that transmits the green laser light from the console to a patient. With some embodiments, OR environment 200 could be provisioned with multiple laser energy consoles 246 (e.g., a morcellator and stone dusting console, or the like). Further, although not shown, OR environment 200 could include other consoles appropriate for the procedure to be performed in OR environment 200.

Laser energy console 246 can include a laser generator 248 and an optical coupler 250, both disposed in a housing 252. The laser generator 248 can be configured to generate laser energy appropriate for treating a target tissue (e.g., stone 280). A treatment fiber 254 (e.g., one of therapy devices 114, or the like) can be coupled to the laser generator 248 via the optical coupler 250. In some embodiments, laser generator 248 can comprise multiple light sources (e.g., a treatment beam, multiple treatment beams, an aiming beam, a diagnostic beam, etc.). Further, laser generator 248 can often include various optical components and sensors configured to measure characteristics or qualities of the laser energy and its effect on the stone 280, or adjacent tissue.

Laser energy console 246 can include computing system 256 (e.g., like computer system 124) which itself can include a display 258 (e.g., touch screen display, or the like). Laser energy console 246 can also include input and/or output devices (not shown), such as, buttons, lights, switches, foot pedals, etc.

Housing 252 can include an interface (not shown) with connection sockets and/or busses to which computing system 256 can be communicatively coupled to centralized operating theater controller 122 via IT infrastructure 120. Such interface can also couple laser energy console 246 to a source of power via operating room infrastructure 118. For example, a connection cable (not shown) could couple computing system 256 to centralized operating theater controller 122 in endoscope console 206 (e.g., via IT infrastructure 120, or the like) and couple laser energy console 246 to power provided by operating room infrastructure 118.

During an example lithotripsy procedure, the treatment fiber 254 can be inserted into port 244 of endoscope handles 208 and pushed through a working channel (not shown) of elongated shaft 214 such that a distal end 260 of the treatment fiber 254 can be positioned proximate to stone 280 in urinary system 202. For example, graphical element 226a depicts an image captured by camera 218 of endoscope 204 in which the distal end 260 of the treatment fiber 254 and stone 280 are shown in the urinary system 202. Laser generator 248 can generate laser energy, which is optically coupled to treatment fiber 254 via the optical coupler 250. The laser energy is conveyed through the treatment fiber 254 and emitted from the distal end 260, where it may be incident on stone 280 to cause the stone 280 to be treated (e.g., ablated, fragmented, dusted, etc.

The computing system 256 can control laser generator 248 (e.g., responsive to input from endoscope 204, endoscope handle 208, responsive to an input device like a foot pedal, responsive to sensor(s) output, responsive to control signals from centralized operating theater controller 122, or the like) to cause the laser generator 248 to generate laser energy having parameters appropriate for the treatment to be generated. Examples of this are described in greater detail below.

In some embodiments, the working channel in which the treatment fiber 254 is inserted is different from the working channel through which fluid supplied by fluidics unit 228 flows. With some embodiments, the working channel in which the treatment fiber 254 is inserted is the same working channel through which fluid supplied by fluidics unit 228 flows.

A user (e.g., physician, a nurse, an assistant, or the like) of OR environment 200 can configure (e.g., enter treatment therapy details, or the like) via the computing components of each respective one of therapy consoles 112 provisioned in OR environment 200. For example, a user can configure endoscope 204 via computing system 212, configure fluidics unit 228 via computing system 234, and configure laser energy console 246 via computing system 256. As another example, a user can configure individual ones of the components of OR environment 200 via centralized operating theater controller 122.

Further, a user can perform a treatment via one or more of the therapy devices 114 described above. For example, endoscope 204 includes endoscope handle 208, which is depicted in use by a user 282 in FIG. 2F. As outlined above, the endoscope handle 208 can be fluidly coupled to fluidics unit 228 via cassette and tubing set 242. Further, a treatment fiber 254 can be disposed through endoscope handle 208 and into urinary system 202. It is to be appreciated that although FIG. 2F depicts a user 282 manipulating endoscope handle 208 during a procedure in OR environment 200, other embodiments may provide robotic, non-manual, or non-touch-based control of devices, such as, endoscope handle 208. Further, it is to be appreciated that the user need not be in the operating theater to control the therapy consoles 112 (e.g., see FIG. 3A).

In some embodiments, the endoscope 204 may include one or more sensors, which can be disposed proximate the distal end 216 of the elongated shaft 214. For example, FIG. 2F depicts pressure sensor 262 at the distal end 216 of the elongated shaft 214. Pressure sensor 262 can be configured to measure an intraluminal pressure (ILP) within the treatment site (see FIG. 2A). The endoscope 204 may also include other sensors such as, for example, a temperature sensor 264, a grating 266 (e.g., a Fiber Bragg grating, or the like) to detect stresses, and/or an antenna or electromagnetic sensor 268 (e.g., a position sensor).

Further, as noted, the endoscope 204 includes at least one camera 218 disposed at the distal end 216 of the elongated shaft 214 to provide a visual feed (e.g., as shown in graphical element 226a, or the like) to the user. The endoscope handle 208 can have a fluid flow on/off switch 270, which allows the user 282 to control when fluid is flowing through the elongated shaft 214 and into the treatment site. The endoscope handle 208 may further include other buttons 272 that perform other functions (e.g., control other devices provisioned in OR environment 200, or the like). For example, in some embodiments, the endoscope handle 208 may include buttons 272 to control the temperature of the fluid. In some embodiments, the endoscope handle 208 may also include a drainage port 274, which may be connected to a drainage system (e.g., of operating room infrastructure 118) and can be configured to provide a path for return flow of fluid from the treatment site.

As indicated above, all components of the urological OR suite need not reside in the operating theater. For example, equipment and users of OR environment 100 or OR environment 200 could be in different room, buildings, sites, or geographic locations. FIG. 3A depicts an OR environment 300 having multiple rooms. In some embodiments, OR environment 300 can be implemented to provide remote proctorship and/or telesurgery. OR environment 300 provides an advantage in that highly trained specialists can be utilized to observe, supervise, train, troubleshoot, and/or otherwise facilitate procedures across OR suites without having to travel to each suite.

FIG. 3A depicts OR environment 300 with operating theater 304, observation room 306, and remote room 308. In general, operating theater 304 is the room where the patient and the bulk of the equipment used to monitor and treat the patient are located. Observation room 306 can be a room proximate too but separate from operating theater 304. For example, observation room 306 can be outside the sterile field, separated from operating theater 304 by a glass wall or window, or the like. Remote room 308 can be a room in the same building as operating theater 304 and observation room 306, in another building from operating theater 304 and observation room 306, or even in another physical or geographic location (e.g., different facility site, different city, different country, partner facility site, etc.)

Communication and interoperability between the equipment in rooms of OR environment 300 is facilitated by centralized operating theater controller 302. Centralized operating theater controller 302 can be implemented as centralized operating theater controller 302 and can include all the components, structure, and features with which centralized operating theater controller 302 is described and attributed herein. As depicted, centralized operating theater controller 302 is provided as a cloud accessible computing system (e.g., in communications network 142). However, centralized operating theater controller 302 could be provided as part of endoscope 204 like described above in FIG. 2B, or any other piece of equipment in OR environment 300.

Operating theater 304, observation room 306, and remote room 308 can be connected via IT infrastructure 120, which can include communications network 142. As such, centralized operating theater controller 302 can communicate with equipment in each room.

OR environment 300 is described with reference to the OR environment 200 described above for consistency and clarity. However, OR environment 300 could be provisioned with equipment other than described herein. Continuing with the example lithotripsy procedure described above, operating theater 304 can include patient bed 310, patient monitor 312, endoscope 204, fluidics unit 228, laser energy console 246, and audio-visual communication equipment 316a (A/V equipment). Operating theater 304 can also be provisioned with theater display 222, equipment controls 314a, and/or robotic devices 108. However, OR environment 300 could be implemented where 304 is not provisioned with theater display 222 and equipment controls 314a, for example, where users needing theater display 222 and equipment controls 314a are not located in operating theater 304. As another example, operating theater 304 could be provisioned with robotic devices 108 where control of equipment in operating theater 304 (e.g., endoscope 204, fluidics unit 228, laser energy console 246, or the like) from remote room 308 is implemented.

Remote room 308 can include remote computing system 318 (e.g., like computer system 124, or the like) including remote display 320 and A/V equipment 316b. Remote room 308 can further include equipment controls 314b. For example, where remote room 308 is used to control (e.g., telesurgery, or the like) equipment in operating theater 304, remote room 308 can include equipment controls 314b. Observation room 306 can include A/V equipment 316c and may also include equipment controls 314c and/or observation display 322.

Equipment controls 314a, 314b, and 314c can include any of equipment controls 116 described herein. A/V equipment 316a, 316b, and 316c can be cameras, microphones, or other equipment configured to provide a view of the procedure and/or communication between rooms. For example, A/V equipment 316a, 316b, and 316c can include a microphone and speaker (e.g., fixed in place in the respective room, wearable, etc.) to permit audio communication between rooms. A/V equipment 316a can include a camera positioned to provide a view of the patient and endoscope handle 208, treatment fiber 254, cassette and tubing set 242, and/or other therapy devices 114. With some embodiments, A/V equipment 316b and 316c can include a camera arranged to provide views of the occupants of each respective room.

Further, various therapy devices 114 can be provided in operating theater 304, for example, endoscope handle 208, cassette and tubing set 242, and treatment fiber 254 can be provided in operating theater 304.

During operation, centralized operating theater controller 302 can provide for monitoring, parameter adjustment, and/or control of equipment in operating theater 304 by users in operating theater 304, observation room 306, and/or remote room 308. For example, centralized operating theater controller 302 can provide monitoring of patient bed 310, patient monitor 312, endoscope 204, fluidics unit 228, and laser energy console 246 via observation display 322 in observation room 306. Further, centralized operating theater controller 302 can provide control of endoscope 204, fluidics unit 228, and laser energy console 246 via equipment controls 314b in remote room 308. For example, a specialist physician can be assigned the responsibility of controlling endoscope 204 via the endoscope handle 208, fluidics unit 228, and laser generator 248, while a nurse can be positioned in operating theater 304 and assigned the responsibility of monitoring patient bed 310 and patient monitor 312. As such, centralized operating theater controller 302 can be configured to control (e.g., robotic devices 108, or the like) equipment in operating theater 304 from inputs and/or control signals received from equipment controls 314b in remote room 308.

FIG. 3B illustrates the centralized operating theater controller 302 shown in FIG. 3A in greater detail. It is to be appreciated that this figure may not depict all elements of centralized operating theater controller 302. For example, elements such as the operating system 144, interconnects, or the like are omitted for clarity. Centralized operating theater controller 302 includes at least a processor 132 and memory storage device 134. In general, memory storage device 134 stores instructions (e.g., application instructions 146, or the like) executable by the processor 132, which when executed cause the centralized operating theater controller 302 to provide remote proctoring and/or telesurgery functionality of any OR environment described herein.

As mentioned, various embodiments herein provide centralized urology operating room control systems that address technical problems including equipment proliferation and spatial constraints in modern urological procedures. The disclosed embodiments illustrate examples of centralized urology operating room control systems that encompass multiple architectural approaches. The embodiments include centralized towers housing multiple therapy consoles, patient support apparatus (e.g., OR tables, surgical tables, and/or patient beds) with integrated recesses configured to receive therapy consoles, and underfloor configurations with access floors creating inner floor spaces for equipment housing. As detailed herein, the centralized urology operating room control systems provide various technical benefits including improved space utilization by reducing equipment footprints within operating rooms. Additionally, the systems herein can provide enhanced noise management through built-in noise cancelling capabilities that minimize distractions and allow medical personnel to focus on centralized notifications, reduced electromagnetic interference by centralizing equipment and containing electromagnetic fields, and enhanced visibility that allows circulating staff to focus attention on necessary information while maintaining line of sight to the patient bed. The system fundamentally addresses the untenable burden of configuring, monitoring, and controlling multiple independent equipment systems that results from the broad array of technological capabilities required for advanced urological procedures by providing unified control interfaces and centralized management of multiple therapy modalities.

The centralized urology operating room control systems comprise a centralized urology control assembly as a core architectural component. As used herein, a centralized urology control assembly refers to a housing-based system that physically consolidates control and power management functions for multiple therapy consoles used in urological procedures. For instance, the centralized urology control assembly includes a housing that serves as the structural foundation for the integrated system. The housing includes a plurality of receptacles which collectively house various consoles and/or equipment described herein, including but not limited to therapy consoles selected from prostate treatment consoles, stone treatment consoles, table-top laser therapy consoles, and endoscope control consoles. For example, the centralized urology control assembly can house a communications console comprising audio output capabilities and wired/wireless connectivity, a centralized processor configured to provide a single user interface via a display (e.g., one or more displays on which the same or similar single user interface is displayed) for controlling operation of the plurality of therapy consoles, and a power input console configured to provide single-source power distribution, among other possible components such as fluidics control equipment, surgical navigation consoles, and noise cancelling consoles. Each of the components in the centralized urology control assembly can be communicatively coupled in a wired and/or wireless manner, facilitating coordinated operation and centralized control of multiple urological therapy systems through unified interfaces that eliminate the need for medical personnel to operate separate control systems for each piece of equipment

The housing can be a modular housing which is configured in a tower or other arrangement. For instance, the housing can include a plurality of modular receptacles which can be preconfigured to receive a respective therapy console of the plurality of therapy consoles and/or various equipment, as detailed herein. For example, each of the receptacles can be sized and shaped to have a volume that is substantially equal to the size and shape of a particular type of therapy console (e.g., a stone treatment console). In some embodiments, each of the receptacles can include a power source (e.g., socket, etc.) that is configured for the particular type of therapy console. For instance, the power source in a given receptacle can have power characteristics that are tailored to the particular voltage or other power requirement of the particular type of therapy console. In some embodiments, one or more of the power sources in the receptacles can be color-coded, shaped, or sized, to fit a corresponding power blade and/or electrical plug of a particular type of therapy console.

The communications console can include a speaker, a microphone, a wireless communication component, a wired communication component, or any combination thereof. For instance, the speaker can be configured to emit audio outputs such as audible notifications and/or alarms that facilitate centralized noise management and allow medical personnel to focus on centralized notifications rather than managing audio feedback from multiple independent systems. For example, an audio alert can be emitted when one or more monitored physiological parameters of a patient are outside of a threshold range, including pressure, fluid flow, energy, or power measurements from therapy consoles, among other possible types of alerts. The microphone can be configured to receive audio inputs such as voice commands that provide hands-free control capabilities during sterile procedures. The voice command can be used to control operation of various components herein such as control operation of one or more of a plurality of therapy consoles, providing the capability for medical personnel to maintain procedural focus while adjusting system parameters. Alternatively, or in addition, in some embodiments, control of the various components herein such as plurality of therapy consoles can be based on inputs provided via a mobile computing device (e.g., a tablet) that is communicatively coupled via the centralized urology control assembly to the plurality of therapy consoles, as described herein. This multi-modal control approach e.g., permitting voice control and/or mobile device control of monitoring and operation of each of the plurality of therapy consoles desirably addresses various technical problems including the challenge that each therapy console and/or endoscope typically has its own custom computing hardware configuration and display, creating what would otherwise be an untenable burden for medical personnel to manage multiple independent therapy console and/or endoscope control systems.

In some embodiments, the communications console can be configured to provide a wired communication capability (e.g., via a wired communication component such as a port, cable interface, or direct connection system). In some embodiments, the communications console can be configured to provide a wireless communication capability (e.g., via a wireless transmitter and/or receiver configured to operate in accordance with a wireless transmission protocol such as a Wi-Fi protocol, BLUETOOTH protocol, cellular communication protocol, and/or another wireless protocol). In some embodiments, the communications console can be configured to provide both wired and wireless communication capability, allowing for flexible connectivity options based on operational requirements and infrastructure constraints. For instance, the communications console can provide wired and/or wireless communication between the centralized urology control assembly and other components herein such as a plurality of therapy consoles, a mobile computing device (e.g., a tablet), one or more remote displays, floor-standing equipment, and/or other components of the centralized urology operating room control system. The communications capabilities facilitate coordinated operation and data exchange between distributed system components while maintaining centralized control functionality, thereby reducing the number of cables running across operating room floors and improving overall system organization.

The centralized processor can include a memory (e.g., a non-volatile memory, volatile memory, or hybrid memory configuration) which stores non-transitory instructions and can include a hardware processor that is configured to execute the instructions stored in the memory. The memory and processor configuration provides the computational foundation for system integration and control coordination across multiple therapy consoles. For instance, the centralized processor can execute instructions stored in the memory to cause display of a single user interface, via a display, at least for monitoring operation of the plurality of therapy consoles while simultaneously managing control signals and data processing for multiple system components. That is, the centralized processor can be configured to provide a single user interface, via a display, for monitoring and/or controlling operation of the plurality of therapy consoles through unified control protocols that eliminate the need for separate interfaces for each therapy console. In some embodiments, the display can be a built-in display that is integral to (e.g., is included in a receptacle or other portion of) the centralized urology control assembly. However, in some embodiments, the display can be remote to the centralized urology control assembly. For instance, the remote display can be a display of a mobile computing device (e.g., a tablet), an operating theater display provisioned in the operating theater, and/or an observation room display provisioned in an observation room of the urological OR suite, among other possible displays including external monitoring stations and distributed control interfaces that support multi-room OR configurations.

Notably, the display can be an interactive display in the form of a single user interface which is configured to permit the user to monitor and control operation of any one or more of the plurality of therapy consoles described herein while maintaining comprehensive oversight of the entire system operation. For instance, the single user interface can include one or more graphical representations of adjustable settings of the plurality of therapy consoles that can be adjusted by an operator (e.g., physician) to cause transmission of a signal to the plurality of therapy consoles to effectuate a corresponding setting change in the actual therapy console without requiring direct interaction with individual console interfaces. That is, the systems herein can desirably permit a physician to readily control (e.g., remotely control) any of the plurality of therapy consoles from a single physical location via a single user interface while maintaining full operational control over multiple therapy modalities simultaneously.

The displays herein can display various information described herein such as visual indications of physiological information (e.g., a composite display comprising pressure, fluid flow, energy, and power measurements), graphical representations of various settings and/or controls associated with one or more of the plurality of therapy consoles, real-time status information from multiple system components, and/or other information relevant to the coordinated operation of the centralized urology operating room control system. The display capabilities can include composite displays that are customizable for different rooms within the OR suite, allowing the first composite display for the operating theater to differ from second composite displays for observation rooms based on procedural requirements and user preferences. Additionally, the displays herein can provide visual indications of other physiological information determined from received physiological information, including turbidity, clarity, temperature, procedure states, and target tissue states, thereby providing comprehensive monitoring capabilities for complex urological procedures.

The systems herein can comprise a plurality of therapy consoles configured to perform specific urological procedures and provide targeted therapeutic interventions. The therapy consoles can be analogous to the therapy consoles described herein such as the therapy consoles 112 as described with respect to FIG. 1, which represent capital equipment and/or infrastructure used for delivery of desired treatment or therapy. For instance, the therapy consoles can be selected from a group consisting of a prostate treatment console, a stone treatment console, a tabletop laser therapy console, and an endoscope control console, each configured to address specific clinical applications within urological procedures. The prostate treatment console can comprise morcellation capabilities, such as VersaCut morcellation systems, configured to provide tissue removal and manipulation during prostate procedures with integrated control and monitoring functionality. The stone treatment console can comprise lithotripsy capabilities, including Swiss LithoClast systems, configured to provide comprehensive calculi management through mechanical, laser, and suction capabilities integrated within a single treatment platform. The tabletop laser therapy console can comprise laser energy generation systems, such as Thulium/TMX configurations, configured to generate laser energy for tissue ablation, fragmentation, dusting, or other therapeutic treatments, with integrated optical coupling and energy delivery control. The endoscope control console can comprise visualization equipment configured to operate with endoscope viewing and control systems, including LVE systems, providing integrated camera control, image processing, and display management capabilities. Additional therapy consoles can include fluidic consoles configured to provide fluid inflow and/or outflow, suction, irrigation management, and pressure control capabilities, as well as soft tissue therapy equipment including ablation consoles, resection consoles, biopsy consoles, and cauterization consoles utilizing laser, electrocautery, or radio frequency (RF) cautery technologies. Each of the therapy consoles can be configured to interface with the centralized urology control assembly through standardized communication protocols and power management systems, allowing for coordinated operation and centralized control of multiple therapeutic modalities during complex urological procedures.

In some embodiments, the systems herein can include a noise cancelling console. The noise cancelling console can be configured to actively cancel operational noise associated with operation of one or more of the plurality of therapy consoles. As such, the noise cancelling console is configured to minimize distractions from concurrent operational sounds generated by the plurality of therapy consoles and facilitate medical personnel focus on a medical procedure, a patient, and/or on centralized notifications and/or alarms. In some embodiments, the noise cancelling console can include hardware that is located within a centralized urology control assembly, urology treatment assembly, or both. For instance, the noise cancelling console can be located with the centralized urology control assembly along with a plurality of therapy consoles. Similarly, in some embodiments, the noise cancelling console can be located in the urology treatment assembly (e.g., one or more centralized urology treatment assembly) including a plurality of therapy consoles.

In some embodiments, the systems herein can include fluidics control equipment such as the fluidics control equipment described herein. For instance, in some embodiments, fluidics control equipment can be disposed in one or more of the receptacles in the housing of the centralized urology control assembly. However, in some embodiments, the fluidics control equipment can be located elsewhere, such as being located as free-standing fluidics control equipment and/or being located in a urology treatment assembly.

As mentioned, in some embodiments on or more of the therapy consoles can be included in centralized urology control assembly. However, in some embodiments, the centralized urology operating room control system encompasses architectural configurations where therapy consoles are physically and functionally separated from the centralized urology control assembly. In such embodiments, the urology treatment assembly represents a dedicated housing structure that consolidates therapy-specific equipment while maintaining communicative connectivity to the centralized control assembly. This architectural approach corresponds to configurations where the centralized control tower is separated from a centralized therapy tower, allowing the centralized control tower to be positioned away from the patient while therapy equipment remains in proximity to the patient for enhanced procedural access.

The urology treatment assembly includes essential visualization and communication capabilities through integration of an endoscope control console and a therapy communications console. The endoscope control console provides centralized visualization management with integrated display capabilities and processor integration for real-time procedural monitoring. The therapy communications console facilitates essential interface coordination for treatment modalities, providing the communication infrastructure necessary for coordinating multiple therapeutic interventions during complex urological procedures.

In some embodiments, the modular therapy-specific urology treatment assemblies can each be configured as a dedicated housing unit for one or more individual therapy consoles. This represents a departure from centralized consolidation toward procedure-specific modularity that addresses the technical reality that in some instances different urological procedures require different combinations of therapeutic capabilities.

Each therapy-specific assembly functions as a specialized container optimized for particular therapeutic modalities such as stone treatment, prostate treatment, or laser therapy applications, among different kinds of urological therapies. This modular approach provides flexible operating room configuration capabilities based on specific procedural requirements while maintaining centralized control coordination through the separate control assembly. The modular architecture addresses the problem that each piece of equipment in the operating room needs to be configured prior to the procedure and then monitored and controlled during the procedure, with each piece of equipment typically having its own custom computing hardware configuration and display.

In some embodiments, the therapy-specific assemblies include comprehensive integration capabilities with various laser systems to accommodate the diverse energy delivery requirements of urological procedures. For instance, such configurations can provide communicative coupling capability to floor-standing laser systems. Such embodiments address the practical requirement that certain laser systems, particularly high-power systems, require floor-standing configurations due to their size, power requirements, and cooling infrastructure needs. That is, floor-standing laser integration can accommodate systems that cannot be practically integrated into tabletop configurations while maintaining centralized control coordination. Each therapy-specific assembly maintains communication protocols that interface with floor-standing laser equipment through standardized control interfaces, allowing the centralized control assembly to manage laser parameters, energy delivery settings, and safety protocols regardless of the physical laser system location. Alternative configurations provide therapy-specific assemblies that are couple to tabletop laser systems and integrate fluidics consoles within the same housing structure. This represents a more compact, integrated approach suitable for procedures requiring coordinated laser energy delivery and fluid management capabilities. The integrated approach combines laser energy generation systems with fluidics management capabilities including irrigation control, pressure management, and waste collection functionality within unified therapy-specific assemblies.

In some embodiments, a centralized urology treatment assembly can be configured to house all therapy consoles rather than distributed therapy-specific assemblies. This represents consolidated therapy equipment housing while maintaining separation from the control assembly, providing the benefits of equipment consolidation while preserving the architectural flexibility of separated control and therapy functions. The centralized urology treatment assembly contains multiple therapy consoles (i.e., therapy consoles) including prostate treatment consoles, stone treatment consoles, laser therapy consoles, fluidics control equipment, and endoscope control consoles within a unified housing structure. This configuration maintains communicative connectivity to the separated centralized control assembly, allowing unified control interfaces while consolidating therapy equipment for improved space utilization and cable management.

In some embodiments, one or more of the components of the systems herein can be integrated within existing operating room infrastructure to optimize space utilization and workflow enhancement. The architectural integration addresses space limitations by incorporating assemblies within operating room structural elements including walls, ceilings, floors, patient beds, and/or patient tables.

For instance, wall integration reduces floor footprint and cable management complexity by incorporating assemblies within wall cavities or wall-mounted configurations. This approach provides equipment access while maintaining clear floor spaces for medical personnel movement and equipment positioning. Wall-integrated systems include protective housings and access panels that maintain sterile field compatibility while providing necessary equipment access.

Ceiling integration provides overhead equipment access while maintaining sterile field integrity and reducing cable management requirements. Ceiling-mounted configurations utilize articulating mounting systems that allow positioning adjustments while maintaining equipment security. The ceiling integration approach corresponds to configurations utilizing access floors and inner floor spaces for equipment housing, providing comprehensive infrastructure integration while maintaining accessibility to necessary floor-standing equipment.

Floor integration encompasses underfloor system implementations that utilize false floor installations providing comprehensive infrastructure integration. These systems feature false floors with structural integrity sufficient to support significant weight from patient beds, staff, and heavy medical equipment including laser systems and mobile extracorporeal shock wave lithotripsy systems. As used herein, a false floor refers to an access floor positioned above and spaced a distance from a sub-floor to create an inner floor space between the access floor and the sub-floor. In some embodiments, at least the centralized urology control assembly can be disposed in the inner floor space in the false floor. Additionally, one or more therapy consoles can be disposed within the inner floor space. For instance, each of the therapy consoles can be configured in a centralized urology treatment assembly is disposed in the inner floor space, as detailed herein. Alternatively, one or more (e.g., each of the therapy consoles can be disposed or located elsewhere). For instance, in some embodiments, the centralized urology control assembly can be located within the inner floor space and each of the therapy consoles can be configured as or disposed in centralized urology treatment assembly that is free-standing movable tower on and movable about the access floor. Similarly, in some embodiments the centralized urology control assembly can be located within the inner floor space and each of the therapy consoles can be configured as or disposed in centralized urology treatment assembly that are disposed within a recess in patient bed or a surgical table, as detailed herein.

Floor-integrated systems provide comprehensive infrastructure solutions through specialized connection interfaces that facilitate seamless integration between underfloor equipment and surface-level medical devices during urological procedures. These systems incorporate three distinct types of connection interfaces, each designed for specific equipment compatibility requirements. Display and data floor connections provide high-bandwidth transmission capabilities for video signals and digital data streams from underfloor equipment to surface-level displays and external network infrastructure. Electrical device floor connections support single-use medical devices that require electrical interaction with underfloor controllers, including endoscopic equipment and electromechanical therapeutic devices. Optomechanical device floor connections accommodate medical devices requiring both optical communication pathways, such as laser fiber transmission for energy delivery systems, and mechanical interface capabilities for fluidics management including irrigation control and suction systems.

The floor-integrated systems utilize a plurality of floor plugs strategically positioned within the access floor structure, with each floor plug associated with and controlled by the centralized urology control assembly. These floor plugs are selected from the group consisting of display and data floor plugs configured for transmitting video and digital data from underfloor equipment to displays and external networks, electrical device floor plugs designed for single-use medical devices requiring electrical interaction with underfloor controllers that are electrical in nature, and optomechanical device floor plugs intended for single-use medical devices requiring optical laser fiber transmission and mechanical fluidics interaction with underfloor controllers.

The floor plug systems provide bidirectional data transmission capabilities, supporting both data transmission to underfloor equipment and data reception from underfloor systems, while also accommodating input signals from surface-level devices such as foot pedals and manual control interfaces used during surgical procedures. This bidirectional functionality facilitates real-time parameter adjustment and feedback mechanisms that are essential for precise surgical control during complex urological procedures.

Floor-integrated systems include comprehensive fluid management through plurality of protective covers specifically configured to cover the plurality of floor plugs, providing essential protection against fluid infiltration that could compromise sensitive electronic components. These protective covers maintain sealed environments around connection points while allowing necessary access for device coupling and system operation. The systems incorporate floor drain systems strategically located within the access floor infrastructure, configured to route fluid away from the centralized urology control assembly, the plurality of floor plugs, or both, ensuring that irrigation fluids, biological materials, and cleaning solutions are effectively managed without impacting system functionality.

The floor drain systems connect to site infrastructure including plumbing drainage systems or incorporate mechanisms for biohazardous waste collection in compliance with medical facility requirements. These drainage systems may include flow measurement capabilities to account for fluid volume changes during procedures, supporting differential measurements of fluid absorption when required for clinical monitoring and documentation purposes.

Floor-integrated systems require false floor construction with structural integrity sufficient to support significant weight from patient beds, medical personnel, and heavy medical equipment including laser systems and mobile extracorporeal shock wave lithotripsy systems. The false floor architecture creates inner floor spaces that house centralized control equipment while maintaining accessibility through strategically positioned access panels and connection interfaces.

Patient bed and table integration represents configurations where centralized therapy towers are built into surgical tables or mobile surgical beds that provide comprehensive positioning capabilities. Table-integrated systems maintain sterility through protective drapes, feature unified graphical user interfaces compatible with multiple system configurations, and incorporate built-in equipment documentation capabilities. Mobile surgical bed implementations incorporate therapy and control consoles into operating beds providing comprehensive mobility and positioning capabilities including angular adjustment mechanisms, height adjustment mechanisms, and adjustable device holders.

The systems herein can include a cable management system. The cable management system can be configured to route connections from the centralized urology control assembly to various other components such as a central display screen and/or one or more of the consoles in the urology control assembly. That is, the cable management system can include a track, clip, or other type of cable retention or direction hardware that is configured to organize a plurality of cables. As such the cable management system provides organized cable routing thereby minimizing floor-level cable proliferation and reducing trip hazards for medical personnel.

The integration approach allows scope, irrigation and laser fiber connections to pass through floors, ceilings, or walls to reach patient tables, providing flexible routing options that minimize cable management complexity within the sterile field. These systems may include outlet configurations that allow users to connect tools and equipment closer to patients through equipment built into walls that connect to backend outlets, with external connectors attached to retractors for cable slack management and power cord routing within walls, floors, and ceilings. Moreover, the modular nature of separated assemblies provides enhanced flexibility for different procedural requirements, allowing operating rooms to be configured with appropriate therapy capabilities while maintaining consistent control interfaces and operational procedures. This architectural approach provides comprehensive solutions for modernizing urology operating room configurations across various applications and environmental contexts while maintaining the core benefits of equipment consolidation, improved workflow efficiency, and enhanced patient care delivery.

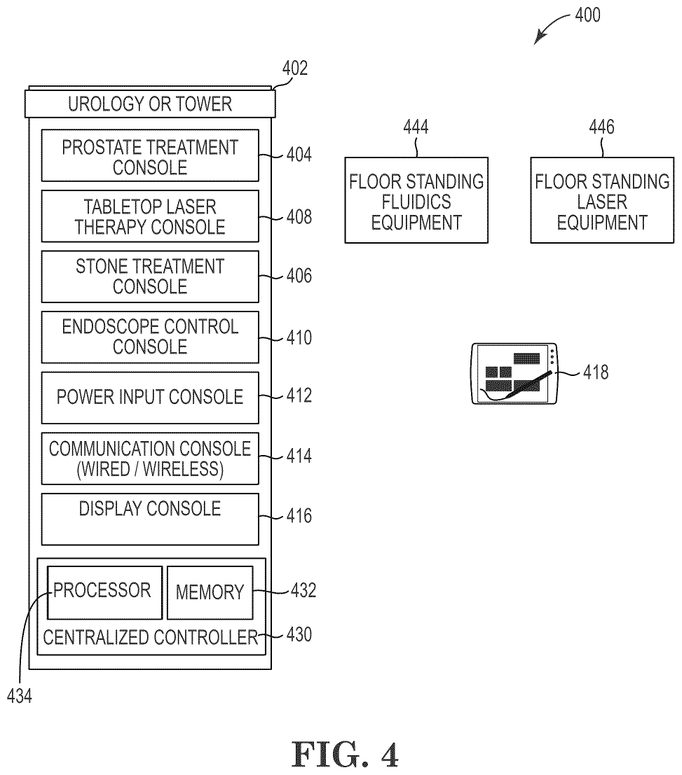

FIG. 4 illustrates a first centralized urology operating room control system 400 in accordance with at least one embodiment. The centralized urology operating room control system 400 comprises a centralized tower approach that consolidates multiple therapy consoles while maintaining separate floor-standing equipment for specific high-power or high-volume applications. As illustrated in FIG. 4, the centralized urology operating room control system 400 includes a centralized urology control assembly 402 that houses multiple integrated therapy consoles and control components within a unified structural framework. The centralized urology control assembly 402 provides space optimization and cable management benefits within the operating room environment while addressing equipment proliferation challenges, as detailed herein.

The centralized urology control assembly 402 includes a housing with a plurality of receptacles that house a plurality of therapy consoles. While various therapy consoles are illustrated in FIG. 4, the centralized urology control assembly 402 can house alternative, additional, or fewer therapy consoles.

For instance, the centralized urology control assembly 402 houses a prostate treatment console 404. The prostate treatment console 404 is configured to provide morcellation capabilities for tissue removal procedures with streamlined control interfaces integrated within the centralized structure. Additionally, the centralized urology control assembly 402 houses a stone treatment console 406. The stone treatment console 406 comprises a single lithotripsy system that incorporates mechanical, laser, and suction capabilities within one integrated platform, thereby eliminating the need for multiple separate stone treatment devices and reducing equipment complexity. Further, the centralized urology control assembly 402 houses a tabletop laser therapy console 408. The tabletop laser therapy console 408 includes laser fiber integration configured to integrate laser fiber directly into endoscope systems, creating unified laser and scope capital equipment that reduces equipment proliferation and improves procedural efficiency through consolidated functionality.

The centralized urology control assembly 402 additionally houses an endoscope control console 410, a power input console 412, a communications console 414, and a display console 416. As detailed herein, the endoscope control console 410 provides centralized visualization management and procedural monitoring capabilities that are integrated within the centralized urology control assembly 402. The power input console 412 offers single-source power optimization for the centralized urology control assembly 402, thereby reducing power cord complexity and providing centralized power management for all integrated components (e.g., each of the integrated therapy consoles). The communications console 414 comprises both wired and wireless communication capabilities, as illustrated in FIG. 4. Additionally, the communications console 414 includes an integrated speaker and microphone for communication hub functionality, facilitating centralized audio notifications and reducing background noise management challenges encountered with multiple independent systems.

The display console 416 is configured for centralized error notifications, status information display, and central screen output capabilities, allowing medical personnel to focus attention on necessary information while maintaining enhanced line of sight to the patient. In some embodiments, the display console 416 includes a built-in display configured to display various patient information, procedural information, and/or information associated with a plurality of the therapy consoles. In some embodiments, the built-in display is configured to concurrently display real-time information associated with two or more of the therapy consoles.

As illustrated in FIG. 4, the system includes a mobile computing device 418. Examples of suitable mobile computing devices include laptops, tablets, and mobile phones. For instance, the mobile computing device 418 can be a tablet. The mobile computing device 418 provides a single user interface control that is operable by various medical personnel such as physicians and/or operating room nurses for managing all integrated therapy consoles within the centralized urology control assembly 402, thereby eliminating the need for separate control interfaces for each therapy console and reducing the cognitive burden on medical personnel.

As mentioned, the centralized urology operating room control system 400 maintains compatibility with various other equipment such as floor-standing laser equipment 446 and floor-standing fluidics equipment 444 that are positioned separate from the centralized urology control assembly 402. The floor-standing laser equipment 446 includes high-power systems such as Holmium lasers and Greenlight lasers that require floor-standing configurations due to size, power requirements, and cooling infrastructure needs that cannot be practically integrated into the centralized urology control assembly 402. The floor-standing fluidics equipment 444 is positioned separate from the centralized urology control assembly 402 and provides suction, aspiration, and/or waste collection capabilities that require high-volume fluid management infrastructure.

The systems herein can include an irrigation bag heater and/or chiller, as detailed herein. For instance, as illustrated in FIG. 4, the system 400 includes an irrigation fluid bag heater 424 integrated within the centralized urology control assembly 402. The irrigation fluid bag heater 424 (and similarly an irrigation bag chiller) provides temperature management of irrigation fluids during urological procedures.

The systems herein can include a surgical navigation console 426. For instance, as illustrated in FIG. 4, the surgical navigation console 426 is integrated within the centralized urology control assembly 402. The surgical navigation console 426 provides real-time procedural guidance and device tracking capabilities during complex urological interventions.

The system 400 desirably provides improved space utilization by consolidating multiple therapy consoles into the centralized urology control assembly 402, promoting better space utilization by medical personnel within the operating room environment. Enhanced noise management is achieved through the centralized location that minimizes distractions from concurrent operational sounds generated by multiple independent systems and allows medical personnel to focus on centralized notifications. In some embodiments, the system 400 is configured to reduce electromagnetic interference. The reduction in electromagnetic interference is at least partly attributable to centralizing the equipment and containing electromagnetic fields within the respective receptacles of the centralized urology control assembly 402, thereby reducing interactions between various equipment and improving overall system performance. In some embodiments, the system 400 is configured to provide improved visibility by allowing circulating staff to focus attention on necessary information (e.g., displayed via a built-in display in the centralized urology control assembly 402 and/or via a remote display), while maintaining enhanced line of sight to the patient bed, enhancing procedural safety and efficiency through the unified control interface approach.

In some embodiments, the system includes a centralized processor 430. The centralized processor 430 includes a memory 432 (e.g., a non-volatile memory, volatile memory, or hybrid memory configuration) that stores non-transitory instructions. The centralized processor 430 includes a hardware processor 434 configured to execute the instructions stored in the memory 432. The hardware processor 434 may include specialized processing capabilities such as central processing units (CPU), graphics processing units, machine learning processing units, or combinations thereof, configured to handle the computational demands of coordinating multiple therapy consoles simultaneously. The memory 432 may comprise computer-readable storage media configured to store various types of data including machine code executable by the hardware processor, graphical instructions and elements for user interface generation, and application instructions for implementing the centralized control functionality described herein. This memory 432 and processor 434 architecture supports the real-time processing requirements for receiving physiological information from multiple therapy consoles, generating composite displays, and providing coordinated control signals across the integrated system components. The memory 432 and processor 434 configuration provides the computational foundation for system integration and control coordination across multiple therapy consoles. For instance, the centralized processor 430 executes instructions stored in the memory to cause display of a single user interface, via a display, at least for monitoring operation of the plurality of therapy consoles while simultaneously managing control signals and data processing for multiple system components.

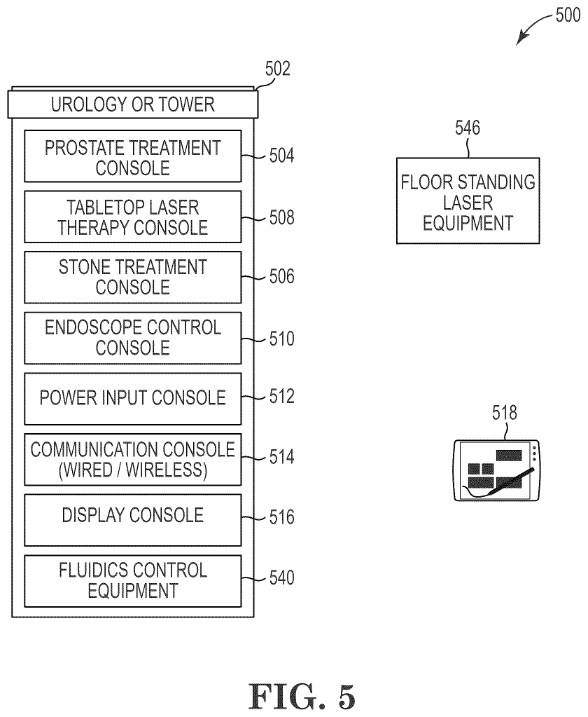

For ease of illustration, the centralized processor is omitted from each of FIGS. 5-15. However, it is noted that each of the centralized urology control assemblies herein can include a centralized processor that is analogous to the centralized processor 430

FIG. 5 illustrates a second centralized urology operating room control system 500 in accordance with at least one embodiment. The system 500 is analogous to system 400 in FIG. 4, with the key distinction being the integration of fluidics control equipment 540 within the centralized urology control assembly 502 rather than utilizing separate floor-standing fluidics equipment.

As illustrated in FIG. 5, the centralized urology control assembly 502 houses a stone treatment console 506, a tabletop laser therapy console 508, an endoscope control console 510, a power input console 512, a communications console 514, and a display console 516. The integrated fluidics control equipment 540 provides irrigation management, suction capabilities, and fluid flow control directly within the centralized urology control assembly 502, eliminating the need for separate floor-standing fluidics equipment.

The system 500 includes a mobile computing device 518 that provides unified control interface capabilities analogous to the mobile computing device 418 described with respect to FIG. 4. Floor-standing laser equipment 546 remains positioned separate from the centralized urology control assembly 502, maintaining compatibility with high-power laser systems that require floor-standing configurations. This configuration permits the fluidics control equipment 540 to be positioned closer to the patient while maintaining the centralized control and power management benefits described with respect to FIG. 4.

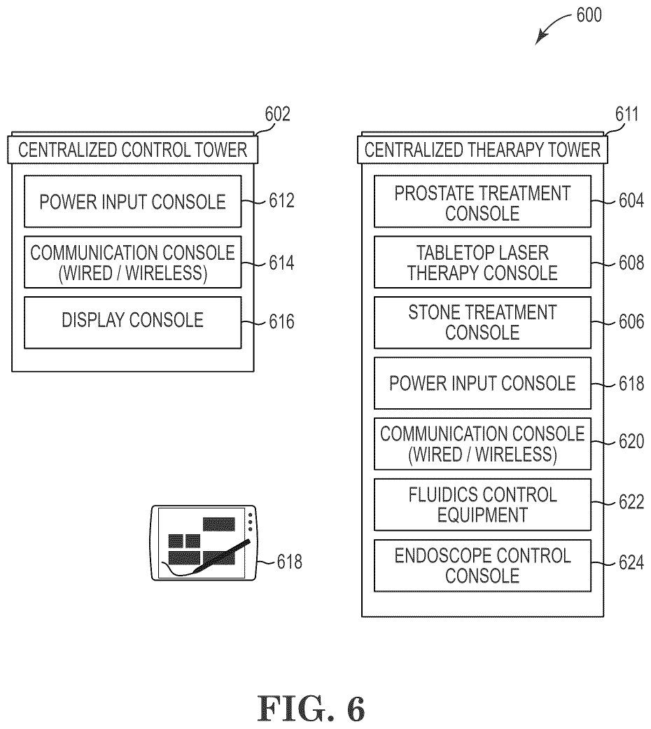

While the embodiments described in FIGS. 4 and 5 demonstrate centralized urology operating room control systems with unified tower configurations, alternative architectural approaches provide enhanced operational flexibility through physical separation of control and therapy functions. The separated tower embodiment illustrated in FIG. 6 maintains the core benefits of centralized control while allowing readily positioning of control interfaces and therapy equipment based on procedural requirements and operator preferences. This configuration addresses the challenge that control functions may be positioned away from the patient for operator convenience while therapy equipment remains positioned for enhanced patient access and procedural efficiency.

FIG. 6 illustrates a third centralized urology operating room control system 600 in accordance with at least one embodiment. The system 600 represents a separated tower configuration that physically divides control functions from therapy functions, allowing the centralized control tower 602 to be positioned away from the patient while the centralized urology treatment assembly 611 remains near the patient for enhanced procedural access.

The centralized control tower 602 houses control-specific components including a power input console 612, a communications console 614 comprising wired and wireless communication capabilities with integrated speakers and microphone functionality, and a display console 616 configured for error notifications and status information display. The centralized control tower 602 serves as the primary command center for managing system operations while being positioned for operator convenience.

The centralized urology treatment assembly 611 houses the therapy-specific equipment including a prostate treatment console 604, a stone treatment console 606, a tabletop laser therapy console 608, an endoscope control console 624, a power input console 618, a communications console 620, and fluidics control equipment 622. This separation allows therapy equipment and fluidics control equipment to remain positioned for optimal access to the patient (e.g., proximate to the patient), while control functions are managed from a dedicated operator location (e.g., at the mobile computing device 618 and/or the centralized control tower 602).

A mobile computing device 618 provides unified interface control across both the centralized control tower 602 and the centralized urology treatment assembly 611, permitting operators to manage the distributed system components through centralized control protocols while maintaining the architectural flexibility of the separated tower configuration. The key distinguishing feature of system 600 is this separation of control and therapy functions, which includes comprehensive lithotripsy capabilities in one rolling capital stack corresponding to the Trilogy/Moses tower design while allowing positioning flexibility based on procedural requirements and operator preferences. The system 600 maintains the unique capability of combining irrigation, laser source and scope capital that can be positioned close to the physician and patient while allowing control functions to be managed from an operator position. This separated architecture allows the control tower 602 to be positioned away from the patient while the therapy equipment in centralized urology treatment assembly 611 remains near the patient, thereby optimizing both operator ergonomics and patient access for enhanced procedural efficiency.

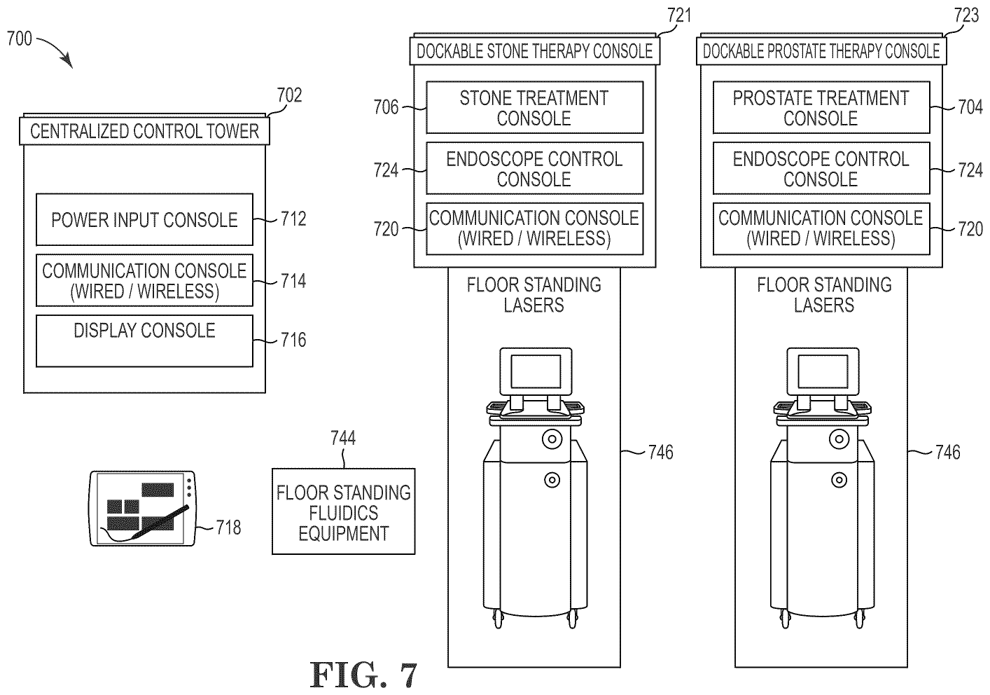

FIG. 7 illustrates a fourth centralized urology operating room control system 700 in accordance with at least one embodiment. The system 700 represents a dockable therapy console configuration that incorporates therapy consoles that can interface with floor-standing laser systems while maintaining separation from fluidics equipment. The key distinguishing feature of system 700 is the dockable therapy console approach that provides modular equipment integration while maintaining compatibility with existing floor-standing capital equipment.

As illustrated in FIG. 7, the centralized urology operating room control system 700 comprises a centralized control tower 702 that includes a power input console 712, a communications console 714, and a display console 716, analogous to the centralized control tower 602 described with respect to FIG. 6. The system 700 comprises a plurality of dockable consoles configured to removably dock with floor-standing equipment, including a dockable stone therapy console 721 and a dockable prostate therapy console 723.

The dockable stone therapy console 721 comprises a stone treatment console 706 with comprehensive lithotripsy capabilities, an endoscope control console 724, and a communications console 720 providing wired and/or wireless connectivity. The dockable prostate therapy console 723 comprises a prostate treatment console 704 configured for morcellation and tissue removal procedures, an endoscope control console, and a communications console 720 providing wired and/or wireless connectivity. Each dockable console is configured to interface with floor-standing laser equipment 746 while maintaining operational independence from floor-standing fluidics equipment 744.

A mobile computing device 718 provides unified interface control for the centralized control tower 702, dockable therapy consoles 721 and 723, and coordination with floor-standing equipment. This dockable modular approach allows individual therapy-specific consoles to be independently positioned and interfaced with existing floor-standing equipment, providing maximum configurational flexibility for different procedural requirements while maintaining centralized control coordination through the mobile computing device 718.

The embodiments illustrated in FIG. 6 and FIG. 7 represent complementary architectural approaches to solving the fundamental technical problem of equipment proliferation and space optimization in urology operating rooms while maintaining operational flexibility and procedural efficiency. FIG. 6 demonstrates a separated tower configuration where the centralized control tower 602 and centralized urology treatment assembly 611 provide distinct functional zones, allowing control functions to be positioned away from the patient while therapy equipment remains in proximity for enhanced procedural access. In contrast, FIG. 7 illustrates a dockable modular approach where individual therapy-specific consoles 721 and 723 can be independently positioned and interfaced with existing floor-standing equipment, providing maximum configurational flexibility for different procedural requirements. Both embodiments maintain centralized control coordination through unified interfaces (mobile computing device 618 in FIG. 6 and mobile computing device 718 in FIG. 7) while offering distinct advantages. For instance, the separated tower approach of FIG. 6 optimizes workflow by creating dedicated control and therapy zones, while the dockable approach of FIG. 7 maximizes equipment utilization and procedural adaptability by allowing independent console positioning. Together, these embodiments demonstrate the scalable nature of the centralized control concept, providing healthcare facilities with architectural options that can be adapted to specific facility constraints, procedural requirements, and existing equipment investments while maintaining the core benefits of reduced equipment proliferation, improved space utilization, and enhanced operational efficiency.

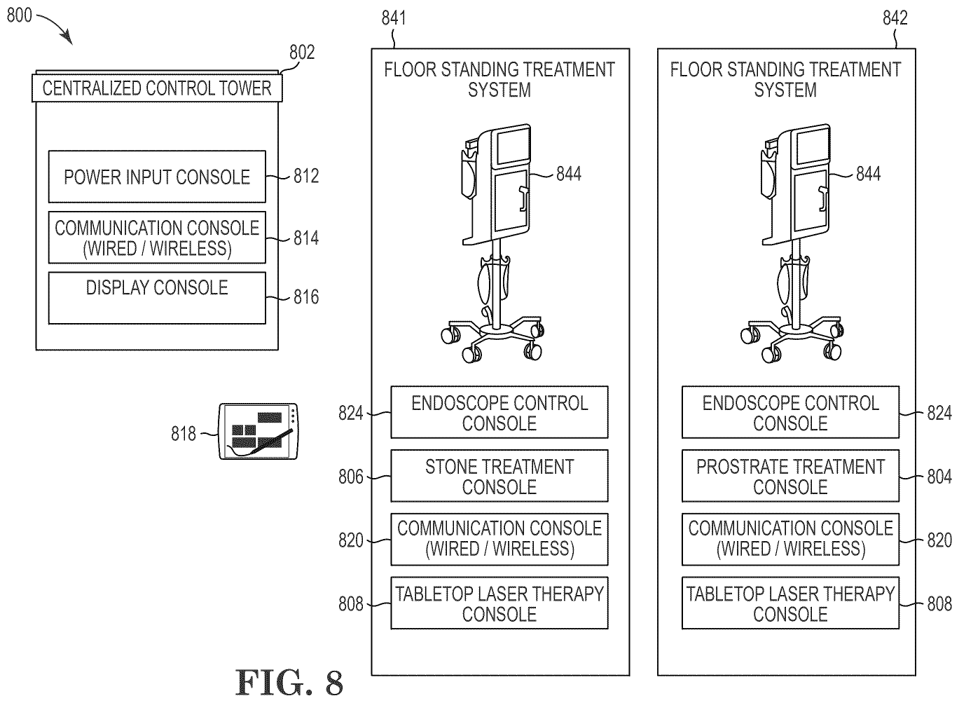

FIG. 8 illustrates a fifth centralized urology operating room control system 800 in accordance with at least one embodiment. The system 800 represents a floor-standing integrated therapy configuration that incorporates tabletop laser systems with integrated fluidics capabilities while maintaining floor-standing treatment systems for enhanced procedural flexibility. The key distinguishing feature of system 800 is the integration of tabletop laser therapy consoles with fluidics control within floor-standing treatment systems, providing comprehensive stone and prostate treatment capabilities in dedicated floor-standing units that can operate independently or in coordination with the centralized control tower 802. That is, as illustrated in FIG. 8, each of the therapy-specific urology specific assemblies are configured to communicatively couple, and/or physically couple to floor-standing fluidics equipment. For instance, each of the therapy-specific urology specific assemblies can be configured to both physically and communicatively couple to floor-standing fluidics equipment.

The centralized urology operating room control system 800 in FIG. 8 comprises a centralized control tower 802. The centralized control tower 802 is analogous to the centralized control tower 702 in FIG. 7. The centralized control tower 802 includes a power input console 812, a communications console 814 providing wired and/or wireless connectivity, and a display console 816 configured to show errors, notifications, and system status information.

The system 800 comprises a plurality of floor-standing integrated treatment systems that combine multiple therapy modalities within single units. The floor-standing treatment systems include a floor-standing stone treatment system 841 and a floor-standing prostate treatment system 843. Each floor-standing treatment system incorporates comprehensive therapy capabilities including endoscope control consoles 824, stone treatment consoles 806 (in the stone configuration) or prostate treatment consoles 804 (in the prostate configuration), communications consoles 820 providing wired and/or wireless connectivity for each of the floor-standing stone treatment system 841 and a floor-standing prostate treatment system 843, and tabletop laser therapy consoles 808 integrated directly within a floor-standing fluidics equipment 844. This integration allows for compact, procedure-specific treatment stations that minimize cable management requirements and optimize workflow efficiency.

In some embodiments, the floor-standing treatment systems 841 and 843 may incorporate image processing capabilities, including the integration of image processing boards and single board computers configured to run combined graphical user interfaces. This enhancement can provide advanced computational capabilities for real-time image analysis and system coordination directly within the treatment units.

A mobile computing device 818 is configured to provide unified interface control for the centralized control tower 802 and the floor-standing integrated treatment systems 841 and 843. The mobile computing device 818 allows operators to manage the distributed system components through centralized control protocols while maintaining the flexibility provided by the integrated floor-standing architecture.

The embodiment illustrated in FIG. 8 demonstrates an integrated floor-standing approach that combines the benefits of modular therapy components with the operational efficiency of integrated systems. By incorporating tabletop laser therapy consoles 808 directly within the floor-standing treatment systems 841 and 843, system 800 eliminates the need for separate laser capital equipment while maintaining the full therapeutic capabilities required for comprehensive urological procedures. This configuration optimizes floor space utilization and reduces equipment proliferation while providing dedicated, procedure-specific treatment stations that can operate independently or in coordination through the centralized control architecture.

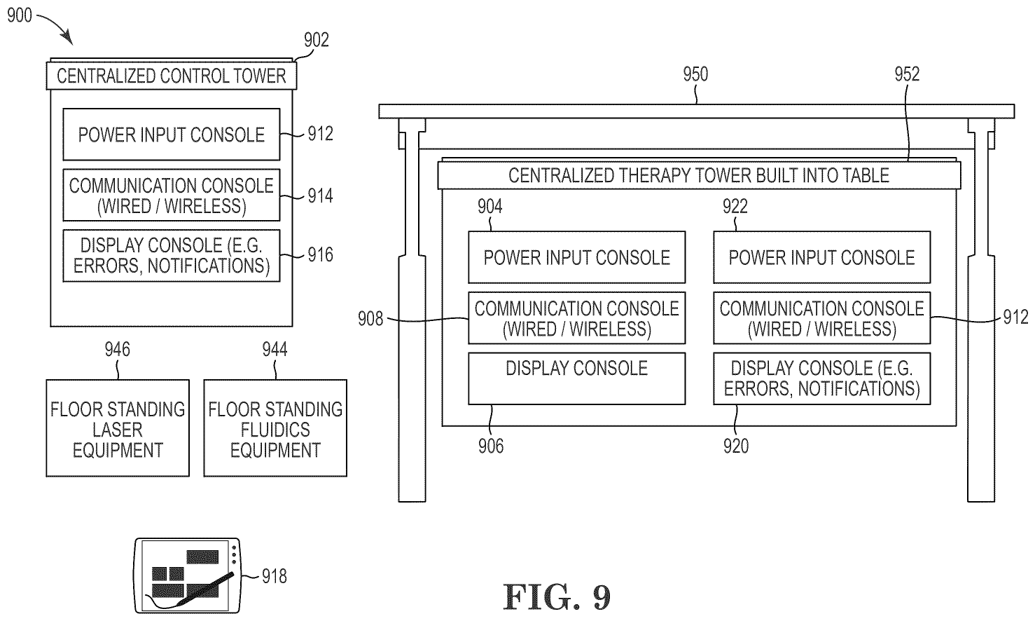

FIG. 9 illustrates a sixth centralized urology operating room control system 900 in accordance with at least one embodiment. The system 900 represents a table-integrated configuration that incorporates all therapy consoles into a surgical table assembly that is routinely used within OR settings. The key distinguishing feature of system 900 is the integration of comprehensive urology treatment capabilities directly into the surgical table infrastructure, providing a centralized therapy platform that maintains sterile field integrity while optimizing workspace utilization and procedural workflow efficiency.

For instance, the centralized urology operating room control system 900 in FIG. 9 comprises a centralized control tower 902. The centralized control tower 902 is analogous to the centralized control tower 802 in FIG. 8. The centralized control tower 902 includes a power input console 912, a communications console 914 providing wired and/or wireless connectivity, and a display console 916 configured to show errors, notifications, and system status information.

The system 900 comprises a centralized therapy tower built into table 950 that integrates multiple therapy modalities within the table structure. The table-integrated treatment assembly 952 includes a plurality of consoles and equipment. For instance, the table-integrated treatment assembly 952 includes a prostate treatment console 904, fluidics control equipment 922, a tabletop laser therapy console 908, a power input console 912, a stone treatment console 906, and a communications console 920 providing wired and/or wireless connectivity for the table-integrated treatment assembly 952 with one or more components such as the centralized control tower 902. This integrated configuration allows the surgical table 950 to serve as both a patient support platform and a comprehensive treatment delivery system, minimizing equipment proliferation while maintaining full therapeutic capabilities.

The centralized therapy tower built into table 950 may be implemented as an attachment to a standard OR surgical table, a standalone system that fits beneath a standard OR table, or as a system fully integrated into a standard OR table structure. The table-integrated embodiment is designed with the ability to remain sterile and protected from fluid contamination through the use of surgical drapes, ensuring compliance with sterile field requirements during urological procedures.

Floor-standing laser equipment 946 and floor-standing fluidics equipment 922 may still be utilized when needed depending on specific procedural requirements. The table-integrated system provides flexibility to accommodate varying procedural complexities while maintaining the core benefits of centralized control and reduced equipment footprint.

A mobile computing device 918 is configured to provide unified interface control for the centralized control tower 902 and the table-integrated therapy consoles. The mobile computing device 918 may incorporate voice control capabilities and remote control functionalities, allowing operators to manage the integrated system through multiple interface modalities while maintaining sterile technique.

The embodiment illustrated in FIG. 9 represents an innovative approach to surgical table design that transforms the patient support platform into an active treatment delivery system. By incorporating therapy consoles directly into the table structure, system 900 eliminates the need for separate mobile equipment while maintaining immediate access to all necessary treatment modalities. This table-integrated approach optimizes sterile field management, reduces cable management complexity. Additionally, the table-integrated approach can provide a built-in barcode scanning capability to document equipment and tool usage throughout the procedure, enhancing both operational efficiency and procedural documentation accuracy.

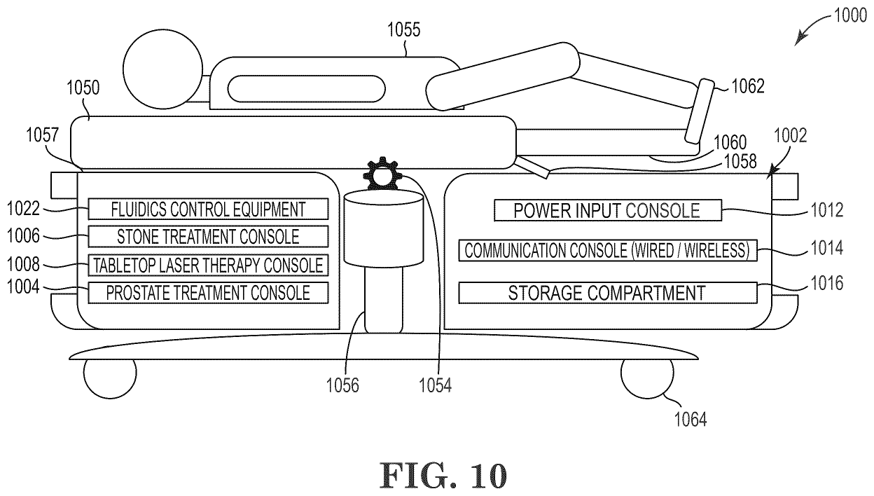

FIG. 10 illustrates a seventh centralized urology operating room control system 1000 in accordance with at least one embodiment. The system 1000 represents a mobile patient support apparatus configuration that incorporates comprehensive therapy and control consoles directly into an operating bed that is mobile and adjustable. The key distinguishing feature of system 1000 is the integration of both therapy capabilities and control functions within a patient support apparatus that provides advanced positioning mechanisms and mobility, creating a self-contained treatment platform that can be repositioned as needed while maintaining full therapeutic functionality.

For instance, the centralized urology operating room control system 1000 in FIG. 10 comprises a centralized control tower 1002. The centralized control tower 1002 is analogous to the centralized control tower 902 in FIG. 9. The centralized control tower 1002 includes a power input console 1012, a communications console 1014 providing wired and/or wireless connectivity, and a display console 1016 configured to show errors, notifications, and system status information.

The system 1000 comprises a mobile patient support apparatus 1050 that integrates multiple therapy consoles 1057 within the patient support structure. The mobile patient support apparatus 1050 includes a patient support surface 1052 and a plurality of integrated consoles 1057 positioned beneath or within the patient support apparatus. The integrated therapy consoles 1057 include a prostate treatment console 1004, a stone treatment console 1006, a tabletop laser therapy console 1008, fluidics control equipment 1022, a communications console 1020 providing wired and/or wireless connectivity, and a storage compartment 1024 for housing additional equipment or consumables.

The mobile patient support apparatus 1050 includes advanced positioning mechanisms to optimize patient positioning and procedural access. The positioning mechanisms include an angular adjustment mechanism 1054 providing electromechanical and/or mechanical capability for tilting a patient 1055 slightly for physiological management and adjustment of gravity-based fluid flows, a height adjustment mechanism 1056 providing electromechanical and/or mechanical capability for vertical adjustment to accommodate preferred physician ergonomics, an adjustable urologic device holder 1058 for positioning and holding devices during procedures while the physician switches steps, an adjustable leg positioner 1060 for positioning and securing patient legs during procedures requiring pelvic region access, and an adjustable foot positioner 1062 that may automatically adjust as the leg positioner is modified.

The mobile patient support apparatus 1050 includes mobility features such as wheels 1064 allowing for repositioning of the surgical bed as needed, and drainage capabilities to remain sterile and protected from fluid contamination through the use of surgical drapes and integrated drainage systems. The apparatus may include structural accommodations such as openings or adjustable consoles to allow for C-arm or ESWL equipment positioning, and integrated connection capabilities allowing systems to plug into the bed with output provisions for display screens.

The mobile patient support apparatus 1050 can communicate via the communication console 1020 with a mobile computing device such as those described herein. As mentioned, the mobile computing device may incorporate voice control capabilities and remote control functionalities, allowing operators to manage the integrated system while maintaining desired positioning relative to the patient and procedure.

The embodiment illustrated in FIG. 10 represents a comprehensive mobile treatment platform that combines patient support, therapy delivery, and system control within a single integrated apparatus. By incorporating therapy consoles directly into the patient support apparatus 1050, system 1000 creates a self-contained treatment environment that can be positioned optimally for different procedures while maintaining immediate access to all necessary therapeutic capabilities. This mobile integrated approach optimizes workflow efficiency by eliminating the need for separate mobile equipment while providing advanced patient positioning capabilities that enhance procedural access and physician ergonomics, all while maintaining centralized control coordination through the integrated system architecture.

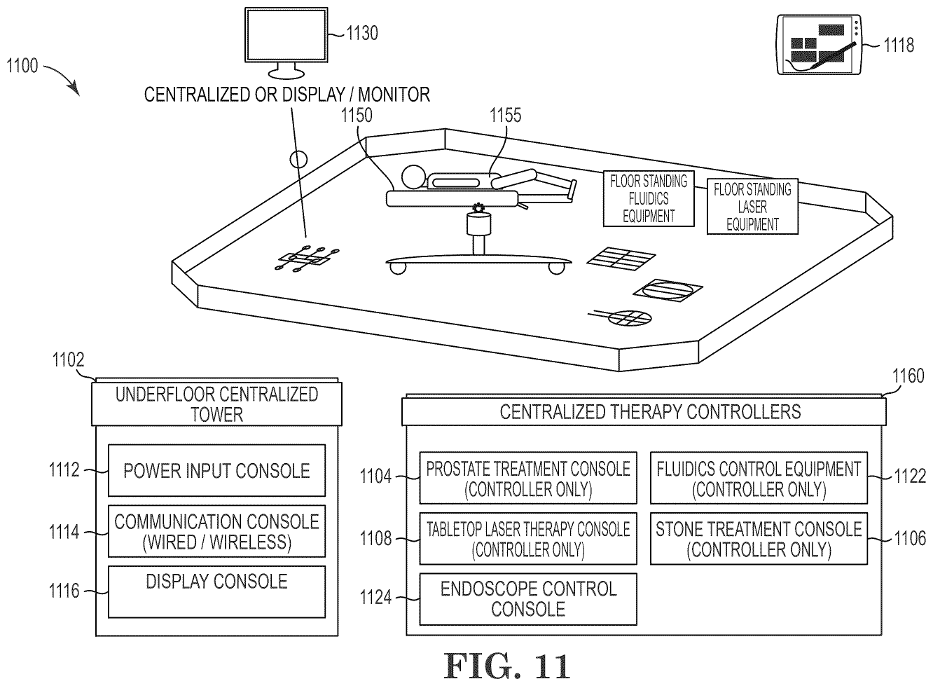

In some embodiments, the centralized urology treatment assembly, the centralized urology control assembly, or both, are configured to be housed within a recess in an operating room structure. Examples of operating room structures includes a wall of the operating room, a ceiling of the operating room, a floor of the operating room, a patient bed, and/or a patient table. For instance, FIGS. 11-15 illustrate examples of centralized urology operating room control systems in which the centralized urology treatment assembly, the centralized urology control assembly, or both, are configured to be housed within a recess in an operating room structure. However, in some embodiments, the centralized urology control assembly, or both, can be configured to be coupled to an exterior surface of the patient support apparatus or can be otherwise located with the OR environment.

FIG. 11 illustrates an eighth centralized urology operating room control system 1100 in accordance with at least one embodiment. The system 1100 represents an underfloor centralized control configuration that places all control equipment underneath the floor while maintaining accessibility to floor-standing treatment equipment. The key distinguishing feature of system 1100 is the complete integration of centralized control functionality within an underfloor infrastructure, creating a sterile operating environment above while housing comprehensive system control capabilities below, thereby maximizing available floor space and minimizing equipment proliferation in the sterile field.

For instance, the centralized urology operating room control system 1100 in FIG. 11 comprises an underfloor centralized control equipment assembly 1102. The underfloor centralized control equipment assembly 1102 is positioned beneath the operating room floor and includes a power input console 1112, a communications console 1114 providing wired and/or wireless connectivity, and a display console 1116 configured to show errors, notifications, and system status information for diagnostic and troubleshooting purposes. That is, the underfloor centralized control equipment assembly 1102 is positioned within an inner floor space under a floor on which a patient support device 1150 and a patient 1155 that is undergoing or scheduled to undergo a urological procedure is located.

Similarly, the system 1100 comprises a centralized urology treatment assembly 1160 integrated within the underfloor centralized control equipment assembly 1102. The centralized urology treatment assembly 1160 includes a prostate treatment console 1104 configured for controller-only functionality, a fluidics control equipment console 1122 configured for controller-only functionality, a tabletop laser therapy console 1108 configured for controller-only functionality, a stone treatment console 1106 configured for controller-only functionality, and an endoscope control console 1124. These controller consoles provide centralized command and control functionality for their respective therapy modalities while the actual therapy delivery components remain accessible in the sterile field above.

In some embodiments, various floor-standing equipment (e.g., floor-standing fluidics equipment and/or floor-standing laser equipment) remain accessible on the operating room floor, providing the physical therapy delivery capabilities required for urological procedures. These floor-standing systems communicate with and are controlled by their respective controller consoles housed within the underfloor centralized control equipment assembly 1102, maintaining operational functionality while reducing the control interface complexity in the sterile field.

A centralized OR display 1130 and a mobile computing device 1118 provide user interface capabilities for the system 1100. The centralized OR display 1130 presents consolidated information from the underfloor centralized control equipment assembly 1102, while the mobile computing device 1118 permits portable control and monitoring of the distributed system components.

The embodiment illustrated in FIG. 11 represents a comprehensive underfloor approach to operating room equipment integration that maximizes sterile field space utilization while maintaining full therapeutic capabilities. By housing all control equipment within the underfloor centralized control equipment assembly 1102, system 1100 eliminates control interface clutter from the sterile field while preserving access to essential floor-standing therapy equipment. This configuration optimizes workflow efficiency by providing centralized control coordination through unified interfaces while maintaining the operational flexibility required for complex urological procedures, all while creating an unencumbered sterile environment that enhances both procedural safety and operational efficiency.

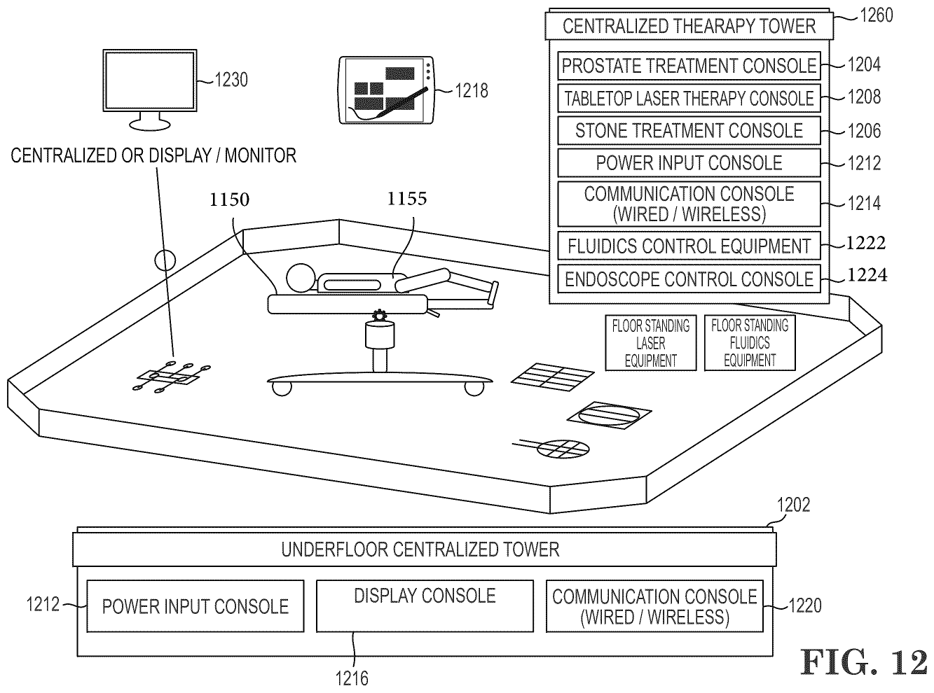

FIG. 12 illustrates a ninth centralized urology operating room control system 1200 in accordance with at least one embodiment. The system 1200 represents an underfloor centralized control configuration that maintains a centralized therapy tower and floor-standing equipment accessible above the operating room floor while housing centralized control equipment underneath the floor. The key distinguishing feature of system 1200 is the combination of comprehensive underfloor centralized control functionality with an accessible centralized therapy tower 1260, providing centralized control coordination while maintaining immediate access to therapy capabilities in the sterile field above.

For instance, the centralized urology operating room control system 1200 in FIG. 12 comprises an underfloor centralized control equipment assembly 1202. The underfloor centralized control equipment assembly 1202 is analogous to the underfloor centralized control equipment assembly 1102 in FIG. 11. The underfloor centralized control equipment assembly 1202 is positioned beneath the operating room floor and includes a power input console 1212, a communications console 1214 providing wired and/or wireless connectivity, and a display console 1216 configured to show errors, notifications, and system status information for diagnostic and troubleshooting purposes.

The system 1200 comprises a centralized therapy tower 1260 that remains accessible on (above) the operating room floor, providing immediate access to multiple therapy capabilities within the sterile field. The centralized therapy tower 1260 includes a prostate treatment console 1204, a tabletop laser therapy console 1208, a stone treatment console 1206, a power input console 1212, a communications console 1220 providing wired and/or wireless connectivity, a fluidics control equipment console 1222, and an endoscope control console 1224. The centralized therapy tower 1260 communicates with and receives coordinated control signals from the underfloor centralized control equipment assembly 1202 while maintaining direct accessibility for procedural use.

Floor-standing laser equipment 1246 and floor-standing fluidics equipment 1244 remain accessible on the operating room floor, providing additional therapeutic capabilities when required for complex procedures. These floor-standing systems maintain operational independence while being coordinated through the underfloor centralized control equipment assembly 1202, allowing for flexible procedural configurations and equipment utilization.

A centralized OR display 1230 and a mobile computing device 1218 provide user interface capabilities for the system 1200. The centralized OR display 1230 presents consolidated information from both the underfloor centralized control equipment assembly 1202 and the centralized therapy tower 1260, while the mobile computing device 1218 provides portable control and monitoring capabilities for the comprehensive distributed system.

The embodiment illustrated in FIG. 12 represents a balance between centralized control architecture and accessible therapy capability integration. By combining the underfloor centralized control equipment assembly 1202 with the accessible centralized therapy tower 1260, system 1200 eliminates control interface complexity from the sterile field while maintaining immediate access to comprehensive therapy capabilities directly within the sterile field. This configuration optimizes workflow efficiency through complete centralized control coordination while providing healthcare practitioners with direct access to essential therapy modalities, creating a comprehensive treatment environment that maximizes both operational efficiency and therapeutic accessibility without compromising sterile field management or procedural workflow.

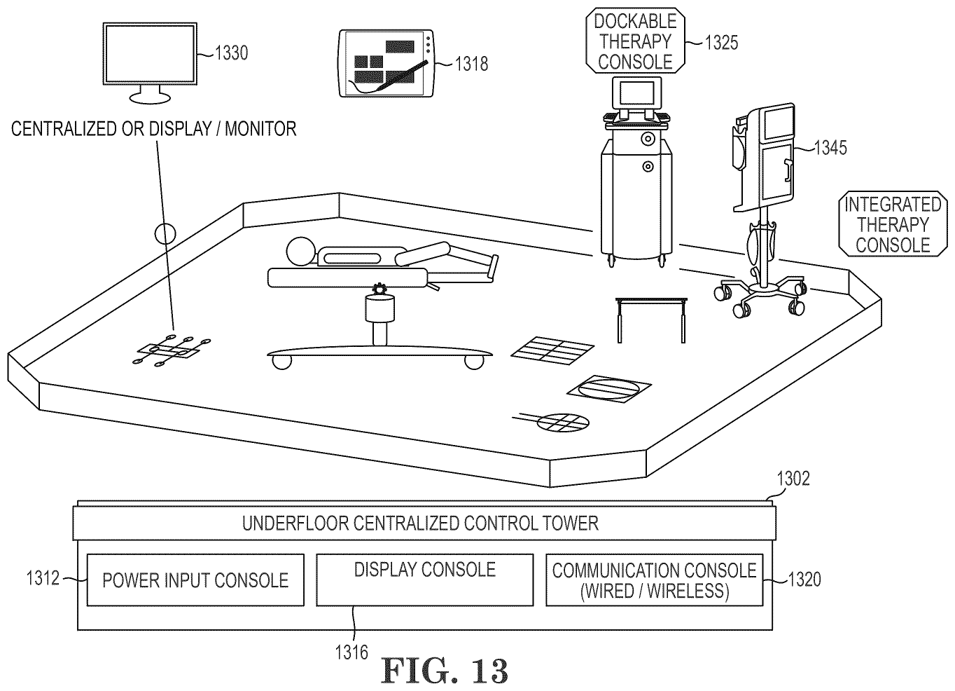

FIG. 13 illustrates a tenth centralized urology operating room control system 1300 in accordance with at least one embodiment. The system 1300 represents an underfloor centralized control configuration that utilizes floor-standing integrated consoles, combining comprehensive underfloor control capabilities with dockable and integrated therapy consoles positioned above the operating room floor. The key distinguishing feature of system 1300 is the integration of complete underfloor centralized control functionality with flexible floor-standing therapy consoles that can provide both dockable and integrated therapy capabilities, creating a comprehensive treatment environment that maximizes both control centralization and therapeutic flexibility.

For instance, the centralized urology operating room control system 1300 in FIG. 13 comprises an underfloor centralized control equipment assembly 1302. The underfloor centralized control equipment assembly 1302 is analogous to the underfloor centralized control equipment assembly 1202 in FIG. 12. The underfloor centralized control equipment assembly 1302 is positioned beneath the operating room floor and includes a power input console 1312, a communications console 1320 providing wired and/or wireless connectivity, and a display console 1316 configured to show errors, notifications, and system status information for diagnostic and troubleshooting purposes.

The system 1300 comprises dockable therapy consoles 1325 and integrated therapy consoles 1345 that remain accessible on the operating room floor while being coordinated by the underfloor centralized control equipment assembly 1302. The dockable therapy consoles 1325 can include therapy-specific consoles configured to interface with floor-standing laser systems while maintaining operational independence from fluidics equipment, similar to the dockable consoles (e.g., 721 and/or 723) described in FIG. 7. The integrated therapy consoles 1345 can include floor-standing treatment systems that combine multiple therapy modalities within single units, incorporating tabletop laser therapy capabilities with integrated fluidics control, similar to the integrated systems (e.g., 841 and/or 842) described in FIG. 8.

The dockable therapy consoles 1325 and integrated therapy consoles 1345 communicate with and receive coordinated control signals from the underfloor centralized control equipment assembly 1302 while maintaining their respective operational characteristics and positioning flexibility. This configuration allows healthcare practitioners to utilize the optimal therapy delivery approach for specific procedures while maintaining complete centralized control coordination through the underfloor architecture.

A centralized OR display 1330 and a mobile computing device 1318 provide user interface capabilities for the system 1300. The centralized OR display 1330 presents consolidated information from the underfloor centralized control equipment assembly 1302, dockable therapy consoles 1325, and integrated therapy consoles 1345, while the mobile computing device 1318 provides portable control and monitoring capabilities for the comprehensive distributed system.

The embodiment illustrated in FIG. 13 represents an optimal integration of underfloor control architecture with flexible floor-standing therapy delivery options. By combining the underfloor centralized control equipment assembly 1302 with both dockable therapy consoles 1325 and integrated therapy consoles 1345, system 1300 eliminates control interface complexity from the sterile field while providing maximum therapeutic flexibility through multiple therapy delivery configurations. This architecture allows healthcare facilities to adapt their equipment configuration based on specific procedural requirements while maintaining the operational efficiency and space optimization benefits of complete centralized control coordination, creating a comprehensive treatment environment that supports both specialized therapy delivery and optimal workflow management.

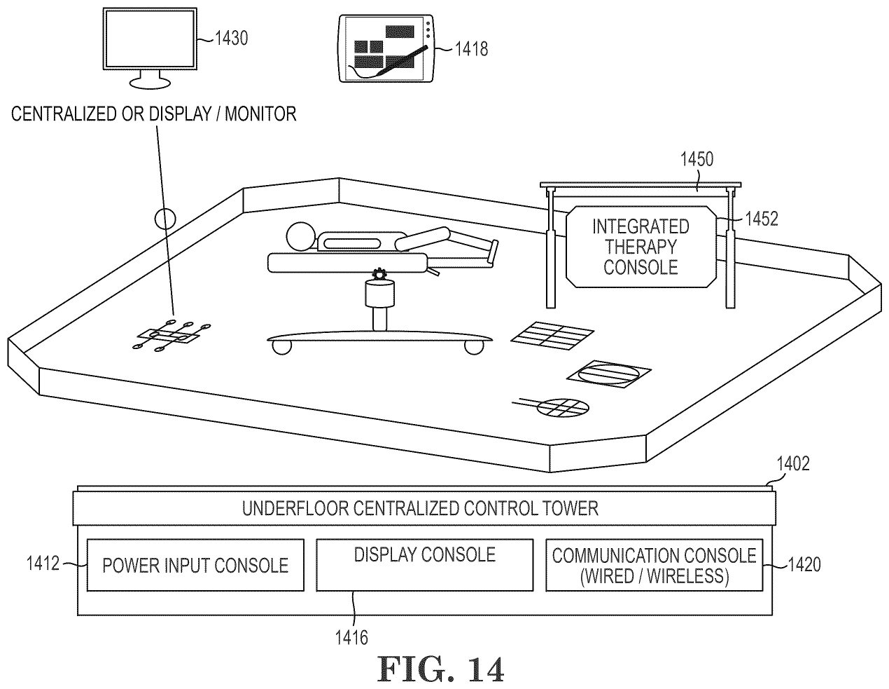

FIG. 14 illustrates an eleventh centralized urology operating room control system 1400 in accordance with at least one embodiment. The system 1400 represents an underfloor centralized control configuration that utilizes the integrated therapy table (e.g., table 950 as illustrated in FIG. 9), combining comprehensive underfloor control capabilities with a table-integrated therapy delivery system. The key distinguishing feature of system 1400 is the integration of complete underfloor centralized control functionality with a surgical table that incorporates integrated therapy consoles directly within the table structure, creating a comprehensive treatment environment that maximizes both control centralization and immediate therapeutic accessibility while maintaining optimal sterile field management.

For instance, the centralized urology operating room control system 1400 in FIG. 14 comprises an underfloor centralized control equipment assembly 1402. The underfloor centralized control equipment assembly 1402 is analogous to the underfloor centralized control equipment assembly 1302 in FIG. 13. The underfloor centralized control equipment assembly 1402 is positioned beneath the operating room floor and includes a power input console 1412, a communications console 1420 providing wired and/or wireless connectivity, and a display console 1416 configured to show errors, notifications, and system status information for diagnostic and troubleshooting purposes.

The system 1400 comprises integrated therapy consoles housed within a surgical table structure that incorporates comprehensive therapy capabilities directly into the table infrastructure, similar to the table-integrated approach described in FIG. 9. That is, a table-integrated treatment assembly 952 includes a plurality of consoles and equipment. For instance, the table-integrated treatment assembly 952 can include various consoles and equipment such as a prostate treatment console 904, fluidics control equipment 922, a tabletop laser therapy console, a power input console, a stone treatment console, and/or a communications console, as detailed herein. This integrated configuration allows the surgical table 950 to serve as both a patient support platform and a comprehensive treatment delivery system, minimizing equipment proliferation while maintaining full therapeutic capabilities.

A centralized OR display 1430 and a mobile computing device 1418 provide user interface capabilities for the system 1400. The centralized OR display 1430 presents consolidated information from both the underfloor centralized control equipment assembly 1402 and the integrated therapy consoles 1460, while the mobile computing device 1418 provides portable control and monitoring capabilities for the comprehensive integrated system.

The embodiment illustrated in FIG. 14 represents the integration of underfloor control architecture with table-integrated therapy delivery, providing maximum space optimization and therapeutic capability consolidation. By combining the underfloor centralized control equipment assembly 1402 with integrated therapy consoles 1460 housed directly within the surgical table structure 1450, system 1400 eliminates control interface complexity from the sterile field while providing immediate access to essential therapy capabilities that are intrinsically linked to patient positioning and support. This configuration optimizes workflow efficiency through complete centralized control coordination while transforming the patient support platform into an active treatment delivery system, creating a comprehensive treatment environment that maximizes both operational efficiency and therapeutic accessibility while maintaining the advanced sterile field management and procedural workflow benefits of the table-integrated approach.

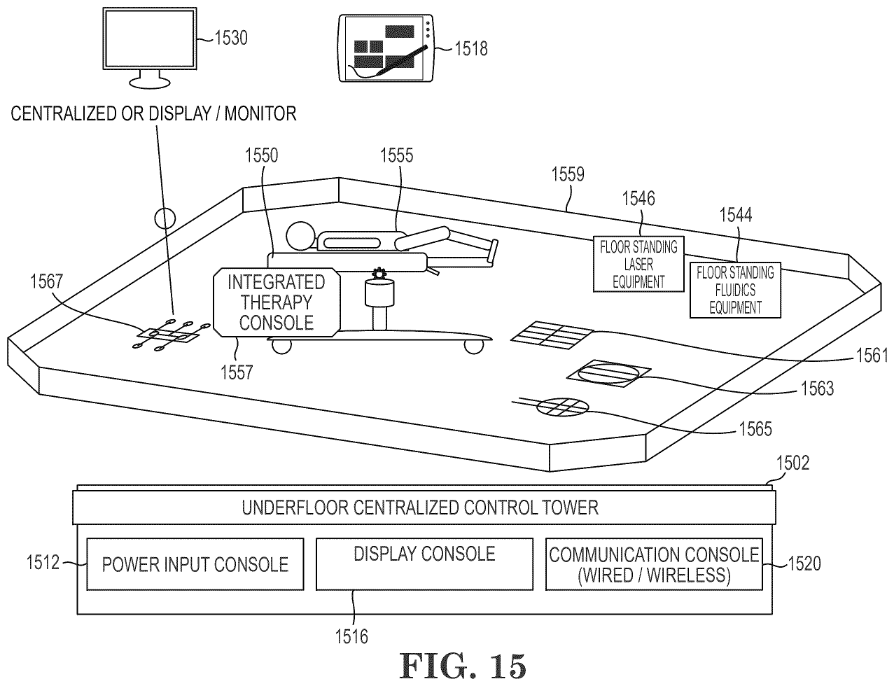

FIG. 15 illustrates a fifteenth centralized urology operating room control system 1500 in accordance with at least one embodiment. The system 1500 represents an underfloor centralized control configuration that utilizes the integrated surgical bed from embodiment #7, combining comprehensive underfloor control capabilities with a mobile patient support apparatus that integrates therapy and control consoles directly into the patient support structure. The key distinguishing feature of system 1500 is the integration of complete underfloor centralized control functionality with a mobile patient support apparatus that provides advanced positioning mechanisms and comprehensive therapy integration, creating a fully integrated treatment environment that maximizes both control centralization and therapeutic accessibility while maintaining optimal patient positioning capabilities.

For instance, the centralized urology operating room control system 1500 in FIG. 15 comprises an underfloor centralized control equipment assembly 1502. The underfloor centralized control equipment assembly 1502 is analogous to the underfloor centralized control equipment assembly 1402 in FIG. 14. The underfloor centralized control equipment assembly 1502 is positioned beneath the operating room floor and includes a power input console 1512, a communications console 1520 providing wired and/or wireless connectivity, and a display console 1516 configured to show errors, notifications, and system status information for diagnostic and troubleshooting purposes.

The system 1500 comprises integrated therapy consoles housed within a mobile patient support apparatus 1550 that incorporates multiple therapy capabilities directly within the patient support structure, similar to the mobile patient support apparatus 1050 described in FIG. 10. The mobile patient support apparatus 1550 includes integrated therapy consoles 1557 such as a prostate treatment console, a stone treatment console, a tabletop laser therapy console, fluidics control equipment, and communications consoles, all housed within or integrated into the patient support apparatus structure. The integrated therapy consoles within the mobile patient support apparatus 1550 communicate with and receive coordinated control signals from the underfloor centralized control equipment assembly 1502, maintaining the benefits of underfloor control architecture while providing immediate access to therapeutic capabilities that are intrinsically linked to patient positioning and support.

Floor-standing laser equipment 1546 and floor-standing fluidics equipment 1544 may remain accessible on the operating room floor when additional therapeutic capabilities beyond those integrated into the mobile patient support apparatus 1550 are required for specific procedures. These floor-standing systems communicate with and receive control coordination from the underfloor centralized control equipment assembly 1502, maintaining operational functionality while keeping the control interface complexity removed from the sterile field.

A centralized OR display 1530 and a mobile computing device 1518 provide user interface capabilities for the system 1500. The centralized OR display 1530 presents consolidated information from both the underfloor centralized control equipment assembly 1502 and the integrated therapy consoles within the mobile patient support apparatus 1550, while the mobile computing device 1518 provides portable control and monitoring capabilities for the comprehensive integrated system.

The embodiment illustrated in FIG. 15 represents the optimal integration of underfloor control architecture with mobile patient support apparatus therapy integration, providing maximum therapeutic capability consolidation with advanced patient positioning functionality. By combining the underfloor centralized control equipment assembly 1502 with integrated therapy consoles housed directly within the mobile patient support apparatus 1550, system 1500 eliminates control interface complexity from the sterile field while providing immediate access to essential therapy capabilities that are directly integrated with comprehensive patient positioning and support mechanisms. This configuration optimizes workflow efficiency through complete centralized control coordination while transforming the patient support platform into a fully mobile, self-contained treatment system that maintains the advanced positioning capabilities and mobility features of the integrated surgical bed approach, creating a comprehensive treatment environment that maximizes both operational efficiency and therapeutic accessibility while providing unparalleled patient positioning flexibility and procedural workflow optimization.

Each of the systems herein such as those illustrated in FIGS. 11-15 can include an access floor positioned above and spaced a distance from a sub-floor to create an inner floor space between the access floor and the sub-floor. The access floor is configured with structural integrity sufficient to support significant weight from patient beds, medical personnel, and heavy medical equipment including laser systems and mobile extracorporeal shock wave lithotripsy systems.

Each of the systems herein such as those illustrated in FIGS. 11-15 can include a plurality of floor plugs strategically positioned within the access floor structure. The plurality of floor plugs can be selected from the group consisting of: display and data floor plugs configured for transmitting video and digital data from underfloor equipment to displays and external networks, electrical device floor plugs designed for single-use medical devices requiring electrical interaction with underfloor controllers that are electrical in nature, such as endoscopes, and optomechanical device floor plugs intended for single-use medical devices requiring optical laser fiber transmission and mechanical fluidics interaction with underfloor controllers. In some embodiments, the plurality of floor plugs can provide bidirectional data transmission capabilities, supporting both data transmission to underfloor equipment and data reception from underfloor systems, while also accommodating input signals from surface-level devices such as foot pedals and manual control interfaces used during surgical procedures

Each of the systems herein such as those illustrated in FIGS. 11-15 can include a plurality of protective covers specifically configured to cover the plurality of floor plugs, providing essential protection against fluid infiltration that could compromise sensitive electronic components. Each of the system in FIGS. 11-15 can include a floor drain system. The floor drain system can be strategically located within the access floor infrastructure, configured to route fluid away from the centralized urology control assembly, the plurality of floor plugs, or both. The floor drain system is sealed and configured to route all fluid away from underfloor electronics. The floor drain system may include flow measurement capabilities to account for fluid volume changes during procedures, supporting differential measurements of fluid absorption when required for clinical monitoring and documentation purposes.

For instance, with continued reference to FIG. 15, the system 1500 can include an access floor 1559 (e.g., a false floor). The access floor 1559 can include a plurality of components such as a floor drain system 1561, an optomechanical floor plug 1563, an electrical floor plug 1565, and a display and data floor plug 1567 (e.g., with is coupled to the centralized OR display/monitor 1530), as detailed herein. The configurations, quantities, and/or locations of the plurality of components in the access floor 1559 can be varied.

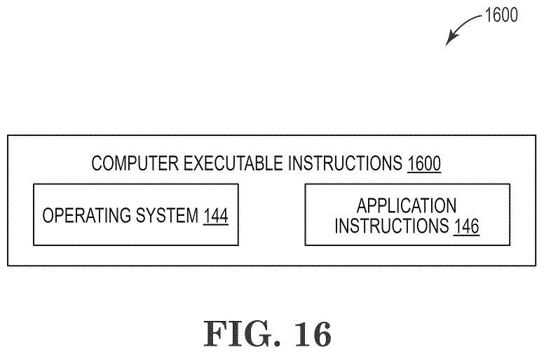

FIG. 16 illustrates computer-readable storage medium 1600. Computer-readable storage medium 1600 may comprise any non-transitory computer-readable storage medium or machine-readable storage medium, such as an optical, magnetic or semiconductor storage medium. In various embodiments, computer-readable storage medium 1600 may comprise an article of manufacture. In some embodiments, computer-readable storage medium 1600 may store computer executable instructions 1602 with which circuitry (e.g., processor 132, or the like) can execute. For example, computer executable instructions 1602 can include instructions to implement operations described with respect to operating system 144, application instructions 146, and/or one or more logical flows. Examples of computer-readable storage medium 1600 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1602 may include any suitable type of code, such as source code, compiled code, interpreted code, executable code, static code, dynamic code, object-oriented code, visual code, and the like.

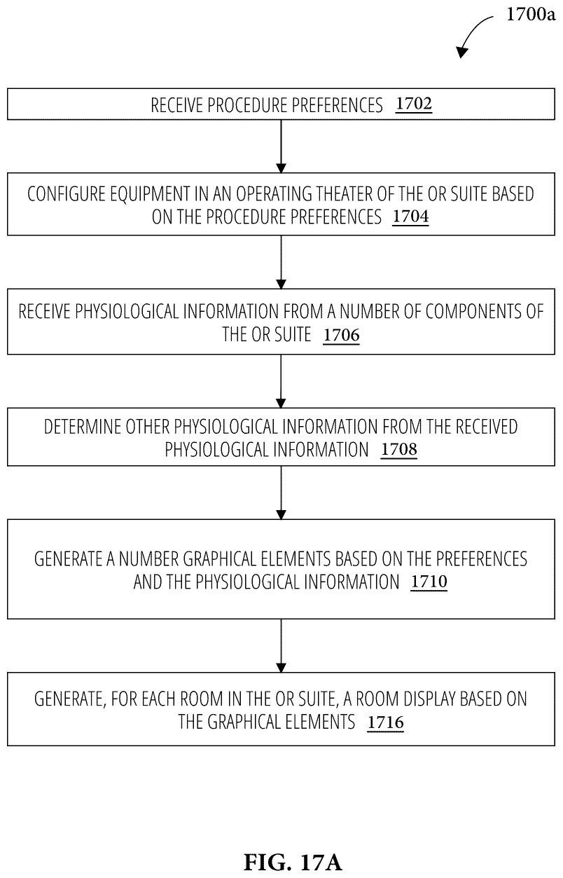

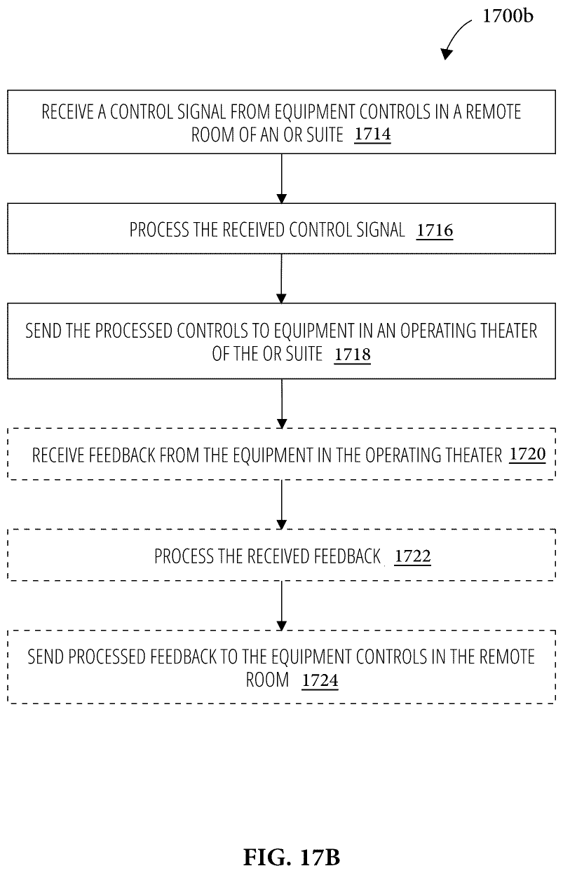

The various operating theater controllers and systems described herein (e.g., 302, or the like) are described with reference to FIG. 17A and FIG. 17B. FIG. 17A and FIG. 17B illustrate logic flows 1700a and 1700b, respectively. These logic flows can be carried out by centralized operating theater controller 302 to provide remote proctoring and/or telesurgery. In some embodiments, centralized operating theater controller 302 can carry out logic flows 1700a and 1700b simultaneously.

Logic flow 1700a can begin at block 1702. At block 1702 “receive procedure preferences” preferences for the treatment procedure being carried out can be received. In some examples, these preferences can be based on the type of procedure and equipment provisioned in the OR suite. In further examples, these preferences can be based on a physician in the OR suite, established clinic preferences, or the like. Processor 132 can execute application instructions 146 to cause centralized operating theater controller 302 to receive procedure preferences 324 (e.g., from a data center, from local storage, from a cloud-based storage location, from equipment controls 314a, equipment controls 314b, or the like). For example, processor 132 can execute application instructions 146 to receive procedure preferences 324 from user (e.g., proctor, physician, etc.) in remote room 308 via equipment controls 314b.

Continuing to block 1704 “configure equipment in an operating theater of the OR suite based on the procedure preferences” equipment in the operating theater of OR environment 300 can be configured based on the procedure preferences. Processor 132 can execute application instructions 146 to cause centralized operating theater controller 302 to configure or otherwise adjust settings on equipment (e.g., endoscope 204, fluidics unit 228, laser energy console 246, etc.) based on the procedure preferences 324. For example, procedure preferences 324 may be parameters for laser energy to be generated by laser energy console 246. As such, processor 132 can execute application instructions 146 to generate configuration control signals 326 and send configuration control signals 326 to laser energy console 246 to cause laser energy console 246 to be placed in a configuration wherein laser energy having the desired parameters will be generated. As another example, procedure preferences 324 may be parameters for fluid flow supplied by fluidics unit 228 and/or ILP. As such, processor 132 can execute application instructions 146 to generate configuration control signals 326 and send configuration control signals 326 to fluidics unit 228 to cause fluidics unit 228 to be placed in a configuration wherein fluid from cassette and tubing set 242 will flow according to the parameters.

Continuing to block 1706 “receive physiological information from a number of components of the OR suite” physiological data can be received from equipment of the OR environment 300. Processor 132 can execute application instructions 146 to cause centralized operating theater controller 302 to receive physiological information 328a from devices and/or consoles of OR environment 300. For example, processor 132 can execute application instructions 146 to receive data (e.g., an information element, sensor output signals, or the like) comprising indications of an intensity of laser energy (e.g., diagnostic energy, aiming energy, treatment energy, or the like) generated by laser generators 248. With some embodiments, processor 132 can execute application instructions 146 to receive physiological information 328a from computing system 256 of laser energy console 246. With some embodiments, processor 132 can execute application instructions 146 to determine the intensity based on signals received from sensors (not shown) of laser energy console 246 where the sensors are configured to measure qualities and/or characteristics of the laser energy.

In some embodiments, processor 132 can execute application instructions 146 to receive physiological information 328a comprising indications of turbidity and/or clarity of scene(s) captured by camera 218 of endoscope 204. In some embodiments, processor 132 can execute application instructions 146 to receive captured scene information 330 comprising image frames of the scene(s). In some embodiments, processor 132 can execute application instructions 146 to receive physiological information 328a comprising indications of an ILP, flow rate of fluidics unit 228, or both.

In some embodiments, processor 132 can execute application instructions 146 to receive physiological information 328a comprising indications of a distance between the distal end 260 of the treatment fiber 254 and the stone 280, a composition of the stone 280, and/or a texture of the stone 280. With some embodiments, laser energy console 246 can be configured to measure this distance and computing system 256 can communicate the distance to centralized operating theater controller 302. With some embodiments, laser energy console 246 can be configured to determine the composition and/or texture of the stone 280 and computing system 256 can communicate the composition and/or texture to centralized operating theater controller 302.

In some embodiments, laser energy console 246 can be configured to measure characteristics of the laser energy and treatment environment (e.g., intensity of laser energy, intensity of reflected laser energy, intensity of autofluorescence emitted responsive to incidence of laser energy on the stone, etc.). Processor 132 can execute application instructions 146 to receive this information as physiological information 328a and derive the distance between the distal end 260 and the stone 280, the composition of the stone 280, and/or the texture of the stone 280 based on physiological information 328a.

In some embodiments, processor 132 can execute application instructions 146 to receive physiological information 328a comprising indications of a size of the stone 280 from endoscope 204. For example, processor 132 can execute application instructions 146 to receive captured scene information 330 where the stone 280 is represented. As another example, processor 132 can execute application instructions 146 to receive radiological image physiological information 328a from radiological imaging devices 106 (e.g., an ultrasound, an x-ray, or the like) where the stone 280 is represented in the radiological image physiological information 328a.

Continuing to block 1708 “determine other physiological information from the received physiological information” other physiological data can be determined (e.g., derived, inferred, or the like) from physiological information 328a received at block 1706. Processor 132 can execute application instructions 146 to cause centralized operating theater controller 302 to derive and/or infer other physiological information 328b from physiological information 328a.

In some embodiments, memory storage device 134 can store models 334. Models 334 can comprise algorithms, functions, and/or trained machine learning (ML) models configured to derive and/or infer physiological information 328b from physiological information 328a.

For example, processor 132 can execute application instructions 146 to determine, using models 334, a turbidity and/or clarity of captured scene information 330 from physiological information 328a and/or captured scene information 330. As another example, processor 132 can execute application instructions 146 to determine, using models 334, a distance between the distal end 260 and the stone 280, a composition of the stone 280, a texture of the stone 280, and/or a size of the stone (e.g., viewed from the camera 218 and/or viewed radiologically) from physiological information 328a, captured scene information 330, and/or radiological image information 332.

Continuing to block 1710 “generate a number of graphical elements from the physiological information” several graphical elements 336 can be generated from the physiological information 328a and 328b (including the captured scene information 330 and radiological image information 332). In general, processor 132 can execute application instructions 146 to generate graphical elements 336 representative of physiological information 328a and 328b associated with each component in the OR suite (e.g., endoscope 204, fluidics unit 228, laser energy console 246, etc.) Further, the graphical elements 336 visually depict represented information using images, text, icons, colors, movement, or the like. With some embodiments, the graphical elements 336 can be like “alerts” or pop-up graphics.

For example, processor 132 can execute application instructions 146 to generate graphical elements 336 comprising an indication of the captured scene information 330. As another example, processor 132 can execute application instructions 146 to generate graphical elements 336 comprising an indication of an ILP and/or flow rate. As another example, processor 132 can execute application instructions 146 to generate graphical elements 336 comprising an indication of a size of the stone 280 or a composition of the stone 280. As another example, processor 132 can execute application instructions 146 to generate graphical elements 336 comprising an indication of an intensity of the laser energy incidence on the stone 280, a distance between the distal end 260 and the stone 280, or the like.

In some embodiments, the information visually represented in graphical elements 336 is based on procedure preferences 324. For example, the information is based on the procedure type and equipment provisions, which can be indicated in procedure preferences 324. As a further example, a first physician (e.g., in operating theater 304) may prefer to pay attention to a first subset of the physiological information 328a and 328b while another physician (e.g., in remote room 308) may prefer to pay attention to a second subset of the physiological information 328a and 328b. In general, the first and second subsets may overlap, but this is not required.

Continuing to block 1712 “generate, for each room of the OR suite, a room display based on the graphical elements” room displays 338 comprising multiple graphical elements 336 can be generated for each room of OR environment 300. For example, a display of room displays 338 can be generated for theater display 222, remote display 320, and/or observation display 322. As outlined above, each of room displays 338 may comprise different combinations of graphical elements 336 depending upon which physical display (e.g., theater display 222, remote display 320, observation display 322, or the like) the display is to be displayed on. In some embodiments, the processor 132 can execute application instructions 146 to generate room displays 338 from graphical elements 336 based on procedure preferences 324. For example, a user of operating theater 304 viewing theater display 222 can specify which graphical elements 336 are preferred or desired to be visible on theater display 222. These preferences can be dictated in procedure preferences 324 and the processor 132 can execute application instructions 146 to generate a display of room displays 338 for theater display 222 from graphical elements 336 based on the procedure preferences 324 for theater display 222. As another example, a user of remote room 308 viewing remote display 320 can specify which graphical elements 336 are preferred or desired to be visible on remote display 320. These preferences can be dictated in procedure preferences 324 and the processor 132 can execute application instructions 146 to generate a display of room displays 338 for remote display 320 from graphical elements 336 based on the procedure preferences 324 for remote display 320.

Logic flow 1700b can begin at block 1714. At block 1714 “receive a control signal from equipment controls in a remote room of an OR suite” control signals from equipment controls in a remote room of an OR suite can be received. Processor 132 can execute application instructions 146 to cause centralized operating theater controller 302 to receive actuation control signals 340 from equipment controls 314b in remote room 308. For example, during operation, a user in remote room 308 can actuate equipment controls 314b and equipment controls 314b can generate actuation control signals 340, which can be communicated to and received by centralized operating theater controller 302 (e.g., via IT infrastructure 120, or the like).

Continuing to block 1716 “process the received control signal” the received control signals can be processed into processed control signals. For example, processor 132 can execute application instructions 146 to process actuation control signals 340 to generate processed control signals 342. In general, processed control signals 342 are versions of actuation control signals 340 configured, processed, or otherwise translated for communication to equipment in operating theater 304 (e.g., robotic devices 108, endoscope 204, endoscope handle 208, fluidics unit 228, laser energy console 246, treatment fiber 254, or the like).

Continuing to block 1718 “send the processed controls to equipment in an operating theater of the OR suite” the processed control signals can be sent to equipment in the operating theater of the OR suite associated with the remote room for which the control signals were received. For example, processor 132 can execute application instructions 146 to send processed control signals 342 to equipment in operating theater 304. For example, actuation control signals 340 may be directed to laser energy console 246 and specify actuation or initiation of laser energy generation. As such, processor 132 can execute application instructions 146 to send processed control signals 342 to laser energy console 246 to cause laser energy console 246 to generate laser energy. As another example, procedure preferences 324 may specify a desire for fluid flow at the treatment site. As such, processor 132 can execute application instructions 146 to send processed control signals 342 to fluidics unit 228 to cause fluidics unit 228 to pump (e.g., via pump 238) fluid through cassette and tubing set 242. With some embodiments, blocks block 1716 and block 1718 can be initiated responsive to receiving a control signal or signals at block 1714.

Logic flow 1700b can optionally include blocks 1720 to block 1724. Where logic flow 1700b includes these blocks, logic flow 1700b can continue from block 1718 to block 1720. At block 1720 “receive feedback from the equipment in the operating theater” feedback signals from equipment in the operating theater of the OR suite can be received. Processor 132 can execute application instructions 146 to cause centralized operating theater controller 302 to receive feedback signals 344 from equipment (e.g., endoscope 204, endoscope handle 208, fluidics unit 228, laser energy console 246, or the like) in operating theater 304.

Continuing to block 1722 “process the received feedback” the feedback signals can be processed into processed feedback signals. For example, processor 132 can execute application instructions 146 to process feedback signals 344 to generate processed feedback signals 346. In general, processed feedback signals 346 are versions of feedback signals 344 configured, processed, or otherwise translated for communication to equipment controls 314b in remote room 308.

Continuing to block 1724 “send processed feedback to the equipment controls in the remote room” the processed feedback signals can be sent to the equipment controls in the remote room of the OR suite. For example, processor 132 can execute application instructions 146 to send processed feedback signals 346 to equipment controls 314b in remote room 308. With some embodiments, blocks block 1722 and block 1724 can be initiated responsive to receiving feedback at block 1720.

As described herein, an OR suite can include multiple displays. For example, the OR environment 200 described above provisioned for a lithotripsy procedure has at least 4 displays, the main theater display 222, the display 220 for the endoscope 204, the display 236 for the fluidics unit 228, and the display 258 for the laser energy console 246. Further, it is to be appreciated that this does not include patient specific devices (e.g., vital monitoring devices, or the like) and anesthesia devices. To that end, the present disclosure provides an OR suite configured to centralize the display of information relevant to the procedure or therapy with which the OR suite is provisioned.

Terms used herein should be accorded their ordinary meaning in the relevant arts, or the meaning indicated by their use in context, unless an express definition is provided, in which case the definition provided herein controls. Additionally, references to “one embodiment” or “an embodiment” do not necessarily refer to the same embodiment. Further, embodiments can be combined where combination does not conflict with the context provided. Words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application.

When the claims use the word “or” in reference to a list of two or more items, that word covers all the following interpretations of the word: any of the items in the list, all the items in the list, and any combination of the items in the list, unless expressly limited to one or the other. When the claims use the word “and/or” in reference to a list of two or more items, that word covers any combination of the listed items. For example, where a claim recites “item 1, item 2, and/or item 3,” the claim means item 1 alone, item 2 alone, item 3 alone, items 1 and 2, items 1 and 3, items 2 and 3, or items 1, 2, and 3.

What is claimed is:

1. A centralized urology operating room control system comprising:
a plurality of therapy consoles selected from a group consisting of a prostate treatment console, a stone treatment console, a tabletop laser therapy console, and an endoscope control console; and
a centralized urology control assembly configured to house at least:
a communications console comprising a speaker;
a centralized controller, wherein the centralized controller includes a memory storing non-transitory instructions and a hardware processor that is configured to execute the instructions to cause display of a single user interface, via a display, at least for monitoring operation of the plurality of therapy consoles; and
a power input console configured to provide single-source power to at least the communications console and the centralized controller.

2. The system of claim 1, wherein the stone treatment console comprises a single lithotripsy system incorporating mechanical, laser, and suction capabilities.

3. The system of claim 1, wherein the plurality of therapy consoles are housed in the centralized urology control assembly.

4. The system of claim 3, wherein the centralized urology control assembly includes a noise cancelling console configured to cancel operational noise associated with operation of one or more of the plurality of therapy consoles.

5. The system of claim 3, wherein the centralized urology control assembly houses fluidics control equipment.

6. The system of claim 1, wherein the plurality of therapy consoles are housed in a urology treatment assembly that is separate from the centralized urology control assembly, wherein the urology treatment assembly includes an endoscope control console and a therapy communication console.

7. The system of claim 6, wherein the urology treatment assembly is comprised of a plurality of therapy-specific urology treatment assemblies which are each configured to house a respective therapy console of the plurality of therapy consoles.

8. The system of claim 7, wherein each of the therapy-specific urology specific assemblies are configured to as communicatively couple, physically couple, or both physically and communicatively couple to a floor-standing laser.

9. The system of claim 7, wherein each of the therapy-specific urology specific assemblies are configured to communicatively couple, physically couple, or both physically and communicatively couple to floor-standing fluidics equipment, and wherein each of the therapy-specific urology specific assemblies includes a table-top laser console.

10. The system of claim 6, wherein the urology treatment assembly is a centralized urology treatment assembly configured to house each of the plurality of therapy consoles.

11. The system of claim 10, wherein the centralized urology treatment assembly, the centralized urology control assembly, or both, are configured to be housed within a recess in an operating room structure, wherein the operating room structure is a wall of the operating room, a ceiling of the operating room, a floor of the operating room, a patient bed, or a patient table.

12. The system of claim 1, wherein:
the single-user interface is configured to concurrently display real-time information associated with two or more of the plurality of therapy consoles via the display; and
the display is a built-in display, a remote display, or both the built in display and the remote display.

13. The system of claim 1, further comprising a surgical navigation console configured to provide:
real-time tracking of at least one of medical devices, body parts, or device motion during urological procedures; and
cause display, via the display, of a representation of the real-time position of the at least one of medical devices, body parts, or device motion during urological procedures.

14. The system of claim 1, wherein the communications console is configured to provide a wired communication capability, a wireless communication capability, or both, with a mobile computing device, wherein the mobile computing device is configured to remotely monitor, control operation of, or monitor and control operation of one or more of the plurality of therapy consoles.

15. The system of claim 1, wherein the communications console includes a microphone, and wherein the centralized processor is configured to control operation of one or more of the plurality of therapy consoles via voice commands received via the microphone.

16. A centralized urology operating room control system comprising:
a plurality of therapy consoles selected from a group consisting of a prostate treatment console, a stone treatment console, a tabletop laser therapy console, and an endoscope control console;
an access floor positioned above and spaced a distance from a sub-floor to create an inner floor space between the access floor and the sub-floor;
a centralized urology control assembly disposed in the inner floor space, the centralized urology control assembly comprising:
a housing defining a plurality of receptacles which collectively house at least:
a power input console providing electrical power to said plurality of therapy consoles;
a communications console comprising a speaker; and
a centralized processor, wherein the centralized processor is configured to provide a single user interface, via a built-in display, for controlling operation of at least one of the plurality of therapy consoles; and
a mobile computing device that is communicatively coupled to and configured to remotely control the plurality of therapy consoles, the centralized urology control assembly, or both the plurality of therapy consoles and the centralized urology control assembly.

17. The system of claim 16, wherein the plurality of therapy consoles are housed in a centralized urology treatment assembly that is separate from the centralized urology control assembly, wherein the centralized urology treatment assembly includes an endoscope control console and a communication console.

18. A centralized urology operating room control system comprising:
a plurality of therapy consoles selected from a group consisting of a prostate treatment console, a stone treatment console, a tabletop laser therapy console, and an endoscope control console;
a patient support apparatus configured to physically support a patient located on the patient support apparatus, wherein the patient support apparatus includes a recess configured to receive at least the plurality of therapy consoles; and
a centralized urology control assembly, the centralized urology control assembly comprising:
a housing defining a plurality of receptacles which collectively house at least:
a power input console providing electrical power to said plurality of therapy consoles;
a communications console comprising a speaker; and
a centralized processor, wherein the centralized processor is configured to provide a single user interface, via a built-in display, for controlling operation of at least one of the plurality of therapy consoles.

19. The centralized urology operating room control system of claim 18, wherein the patient support apparatus is a patient table or a patient bed.

20. The centralized urology operating room control system of claim 18, wherein the centralized urology control assembly is configured to be disposed in the recess in the patient support apparatus or is configured to be coupled to an exterior surface of the patient support apparatus.
